SEQUENCE LISTING (34 sequences)

SEQ ID NO: 1 - DNA, 3952 bp, Sus scrofa
  misc_feature (38)..(38): n is a, c, g, or t
  misc_feature (41)..(41): n is a, c, g, or t
  misc_feature (3637)..(3637): n is a, c, g, or t
  misc_feature (3923)..(3923): n is a, c, g, or t
attgggccgacgtcgcatgctcccggccggccgccatntcngccgcgggaaattcgattggaattcctcgagagcaggaacgtggaaaggagctccggtcccagagcagccaccgcgatgagaggcgctcgccgcgcctgggattttctcttcgtcctgcagctcttgcttcgcgtccagacaggctcttctcagccatctgtgagtccagaggaactgtctccaccatccatccagccagcaaaatcagagttaatcgtcagtgctggcgatgagattaggctgttctgcaccgatccaggatctgtcaaatggacttttgagaccctgggtcagctgagtgagaatactcacgcagagtggatcgtggagaaagcagaggccatgaatacaggcaattatacatgcaccaatgaaggcggtttaagcagttccatttatgtgtttgttagagatcctgagaagcttttcctcgtcgaccctcccttgtatgggaaggaggacaatgacgcgctggtccgctgtcctctgacggacccagaggtgaccaattactccctcacgggctgcgaggggaaaccccttcccaaggatttgaccttcgttgcagaccccaaggccggcatcaccatcaaaaatgtgaagcgcgagtatcatcggctgtgtctacactgctccgccaaccaggggggcaagtccgtgctgtcgaagaaattcaccctgaaagtgagggcagccatcagagctgtacctgttgtggctgtgtccaaagcaagctaccttctcagggaaggggaggaatttgccgtgatgtgcttgatcaaagacgtgtctagttccgtggactccatgtggatcagggagaacagccagactaaagcacaggtgaagaggaatagctggcatcagggtgacttcaattttctgcggcaggaaaggctgacaatcagctcagcaagagttaatgattctggcgtgttcatgtgttacgccaataatacttttggatctgcaaatgtcacaaccaccttagaagtagtagataaaggattcattaatatcttccctatgatgaataccactgtgtttgtaaacgatggagaggatgtggatctaattgttgagtacgaggcgtaccccaaacctgaacaccgacagtggatatatatgaaccgcactgccactgataagtgggaggattatcccaagtctgagaatgaaagtaacatcagatatgtaagtgaacttcacttgaccagattaaaagggaccgaaggaggcacttacacatttctcgtgtccaatgctgatgtcaattcttctgtgacatttaatgtttacgtgaacacaaaaccagaaatcctgactcatgacaggctcatgaacggcatgctccagtgtgtggcggcaggcttcccagagcccaccatcgattggtatttctgtccaggcaccgagcagagatgttccgttcccgttgggccagtggacgtgcagatccaaaactcatctgtatcaccgtttggaaaactagtgattcacagctccattgattacagtgcattcaaacacaacggcacggtggagtgcagggcttacaacgatgtgggcaagagttctgccttttttaactttgcatttaaagaacaaatccatgcccacaccctcttcacgcctttgctgattggttttgtgatcgcagcgggtatgatgtgtatcatcgtgatgattctcacctataaatatctacagaagcccatgtatgaagtacagtggaaggttgtcgaggagataaatggaaacaattatgtctacatagacccaacgcaacttccttatgatcacaaatgggaatttcccaggaacaggctgagttttggcaaaaccttgggtgctggcgccttcgggaaagtcgttgaggccactgcatacggcttaattaagtcagatgcggccatgaccgttgccgtgaagatgctcaaaccaagtgcccatttaacggaacgagaagccctgatgtctgaactcaaagtcttaagttacctcggtaatcacatgaatattgtgaatcttctcggcgcctgcaccattggagggcccaccctggtcattacagaatattgttgctatggtgatctcctgaattttttgagacggaaacgtgattcgtttatttgctcaaagcaggaagatcacgcagaagcggcgctttataagaaccttctgcattcaaaggagtcttcctgcagtgacagtactaacgagtacatggacatgaaacccggagtgtcttatgtggtaccaaccaaggcagacaaaaggagatctgcgagaataggctcatacatagaacgagatgtgactcctgccatcatggaagatgatgagttggccctagacctggaggacttgctcagcttttcttaccaagtggcaaagggcatggccttcctcgcctcgaagaattgtattcacagagacttggcggccagaaatatcctccttactcatggtcgaatcacaaagatttgtgattttggtctagccagagacatcaagaatgattctaattacgtggtcaaaggaaacgctcggctacccgtgaagtggatggcacccgagagcattttcaactgtgtctacacatttgaaagcgatgtctggtcctatgggatttttctgtgggagctcttctctttagggagcagcccctaccctggaatgccagttgattctaaattctacaagatgatcaaggagggtttccgaatgctcagtcctgagcatgcacctgcggaaatgtatgacatcatgaagacttgctgggatgcggatcccctcaaaagaccaacgtttaagcagattgtgcagctgattgagaagcagatttcggagagcaccaatcacatttattccaacttagcgaactgcagcccccaccgggagaaccccgcggtggatcattctgtgcggatcaactccgtgggcagcagtgcctcctccacgcagccgctgcttgtccacgaagacgtctgaagcagaatgggtgtccggggtggggggtgggggggctcctcccccacagcaccggcctactgccattctttttggttttcataatggttattttgtttcccttcaacttgcatcctactccagggtagtggatgctccgctgtaatcctctttacgagcacactttagtggccaatgatttttgtcatcagctgccattgagctgtatatgttcccaatagcacgctagcccccattaacggagagcattcagacttagggaagaggagggtaggacgggctggacaccccaggtccttgacaagtcttctccagtttctgtccaataagtgctgtaatggtttatttgagcacctggctgtcgtcacctccggtccttgtcatcatctgtaacaatatgatgatgatgatgccagaacctaatcccttgatgtggaaaataggatgttaatcaaacaaagggcagaaagaagcctgtgactatctgggctcgagaagtcaagtatttcatgctgggagtaagacgtaagccatggaaaaatgctctccgggcatgaataaggctgctggccatgagcctttttactcctgacctggtttntaagtagtttgttattagggagctggatcggagggaaggcttctgcctgcattttgtatatactcatctataaattgttcatgttcacatatttgaggggggaaaacccgcaaggtgtagtttctggatacaatcctggctcgagtctgctgcgtgtagaaatagctgaagagccagacacgtttgaaggaaacagtgctttttttaagaaaaaaaaaaaaaaaagtcgacatcgatacgcgtggtcaatcactagtgaattcgcggccgcctgcaggtcgaccanaaggagagctcccaacgcgtggagcaagc

SEQ ID NO: 2 - protein, 972 aa, Sus scrofa
MRGARRAWDFLFVLQLLLRVQTGSSQPSVSPEELSPPSIQPAKSELIVSAGDEIRLFCTDPGSVKWTFETLGQLSENTHAEWIVEKAEAMNTGNYTCTNEGGLSSSIYVFVRDPEKLFLVDPPLYGKEDNDALVRCPLTDPEVTNYSLTGCEGKPLPKDLTFVADPKAGITIKNVKREYHRLCLHCSANQGGKSVLSKKFTLKVRAAIRAVPVVAVSKASYLLREGEEFAVMCLIKDVSSSVDSMWIRENSQTKAQVKRNSWHQGDFNFLRQERLTISSARVNDSGVFMCYANNTFGSANVTTTLEVVDKGFINIFPMMNTTVFVNDGEDVDLIVEYEAYPKPEHRQWIYMNRTATDKWEDYPKSENESNIRYVSELHLTRLKGTEGGTYTFLVSNADVNSSVTFNVYVNTKPEILTHDRLMNGMLQCVAAGFPEPTIDWYFCPGTEQRCSVPVGPVDVQIQNSSVSPFGKLVIHSSIDYSAFKHNGTVECRAYNDVGKSSAFFNFAFKEQIHAHTLFTPLLIGFVIAAGMMCIIVMILTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSARIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPHRENPAVDHSVRINSVGSSASSTQPLLVHEDV

SEQ ID NO: 3 - protein, 8 aa, Sus scrofa
PLLVHEDV

SEQ ID NO: 4 - DNA, 936 bp, Sus scrofa
  CDS (1)..(936)
atggccgcgctgctcctgggcgcggtgatgctggtccttcagctccagctggtgccttgccgccccgccatgcccggggccgggccgagccagcaggagcttgtgcggaaagcggcgaccctccaggatgaggtccgggacagcgcggcccccaacggctccgtccagcagctgccgcagaccatcatcatcggcgtgcgcaagggcgggacccgcgcgctgctggagatgctcagcctgcatcccgacgtggctgctgcggagaacgaggtgcacttcttcgactgggaggagcattacagccaaggcctggactggtacctcagccagatgcccttctcctacccgcaccagctcacggttgaaaagacccccgcgtacttcacgtcgcccaaagtgcctgagcgggtccaccgcatgaacccgtccatccggctgctgctcatcctgcgggacccgtcggagcgcgtgctgtccgactacacccaagtgttctacaaccacgtgcagaagcacaagccctacccgtccatcgaggagttcctggtgcgcgacggccgcctcaacgtggactacaaggccctcaaccgaagcctgtaccacgtgcacatgcagaactggctgcgcttcttcccgctgcgccgcatccacatcgtggatggcgaccgcctcatcagggacccttttcctgagatccagaaggtcgagaggttcctgatgctgtcgccgcagatcaacgcctcgaacttctactttaacaaaaccaagggcttttactgcctgcgggacggcggccgggaccgctgcttacatgagtccaaaggccgggcgcacccccagatcgaccccaaactcctcaataaactgcacgaatattttcatgagccaaataagaaatttttcgagcttgtgggcagaacatttgactggcactaa

SEQ ID NO: 5 - protein, 311 aa, Sus scrofa
MAALLLGAVMLVLQLQLVPCRPAMPGAGPSQQELVRKAATLQDEVRDSAAPNGSVQQLPQTIIIGVRKGGTRALLEMLSLHPDVAAAENEVHFFDWEEHYSQGLDWYLSQMPFSYPHQLTVEKTPAYFTSPKVPERVHRMNPSIRLLLILRDPSERVLSDYTQVFYNHVQKHKPYPSIEEFLVRDGRLNVDYKALNRSLYHVHMQNWLRFFPLRRIHIVDGDRLIRDPFPEIQKVERFLMLSPQINASNFYFNKTKGFYCLRDGGRDRCLHESKGRAHPQIDPKLLNKLHEYFHEPNKKFFELVGRTFDWH

SEQ ID NO: 6 - DNA, 1236 bp, Sus scrofa
  CDS (1)..(1236)
atgcggcggcggcgcgctggcagcaggaccatggttgagcgcgccagcaagttcgtgctggtcgtggcgggctcggcgtgcttcatgctcatcctctaccagtacgcgggcccggggctgagcctgggcgcgcccggcggccgcgcgccgcccgacgacctggacctcttccccacgcccgacccgcactacgagaagaagtactacttcccggtgcgcgagctggagcgctcgctgcacttcgacatgaagggcgacgacgtgatagtcttcttgcacatccagaaaacgggcggcaccaccttcggccgtcacctcgtgcagaacgtgcgcctcgaggtgccctgcgactgccggcccggccagaagaagtgcacctgctaccggcccaaccgccgcgagacctggctcttctcccgcttctccacgggctggagctgcggactgcacgccgactggaccgagctcaccaactgcgtgcccggcgtgctggaccgccgcgaccccgccgcgctgcgcacgcccaggaagttctactacatcaccctgctgcgagaccccgtgtcccgctacctgagtgagtggcggcatgtacagcggggggccacatggaagacgtcgctgcacatgtgtgacgggcgcacgcccacccctgaggagctgccaccctgctacgagggcacggactggtcgggctgcacactgcaggagttcatggactgcccctacaacctggccaataaccgccaggtgcgaatgctggccgacctgagcctggtgggctgctacaacctgtccttcatccccgagggcaagcggtcccaactgctgctggaaagcgccaagaagaacctgcggggcatggccttcttcggcctgaccgagttccagcgcaagacgcagtacctgttcgagcggacgttcaacctcaagttcatccggcctttcatgcagtacaacagcacgcgagcgggtggcgtggaggtgggtgaggacaccatccggcgcattgaggagctcaacgacctggacatgcagctgtacgactacgccagggacctcttccagcagcgctatcagtacaagcggcagctggagcgccggcagcagcgcctccggagccgcgaggagcgcctgctgcaccgggccaaggaggcgccacctcggggggacaccgaggagccgggccgagtgcccactgaggactacatgagccacatcatcgagaagtggtag

SEQ ID NO: 7 - protein, 411 aa, Sus scrofa
MRRRRAGSRTMVERASKFVLVVAGSACFMLILYQYAGPGLSLGAPGGRAPPDDLDLFPTPDPHYEKKYYFPVRELERSLHFDMKGDDVIVFLHIQKTGGTTFGRHLVQNVRLEVPCDCRPGQKKCTCYRPNRRETWLFSRFSTGWSCGLHADWTELTNCVPGVLDRRDPAALRTPRKFYYITLLRDPVSRYLSEWRHVQRGATWKTSLHMCDGRTPTPEELPPCYEGTDWSGCTLQEFMDCPYNLANNRQVRMLADLSLVGCYNLSFIPEGKRSQLLLESAKKNLRGMAFFGLTEFQRKTQYLFERTFNLKFIRPFMQYNSTRAGGVEVGEDTIRRIEELNDLDMQLYDYARDLFQQRYQYKRQLERRQQRLRSREERLLHRAKEAPPRGDTEEPGRVPTEDYMSHIIEKW

SEQ ID NO: 8 - DNA, 39 bp, Sus scrofa
gaccacgcgtatcgatgtcgacttttttttttttttttv

SEQ ID NO: 9 - DNA, 33 bp, Sus scrofa
ggaattcctcgagagcaggaacgtggaaaggag

SEQ ID NO: 10 - DNA, 22 bp, Sus scrofa
gaccacgcgtatcgatgtcgac

SEQ ID NO: 11 - DNA, 17 bp, Sus scrofa
gcagcagccacgtcggg

SEQ ID NO: 12 - DNA, 20 bp, Sus scrofa
tcagtgycagtcraatgttc

SEQ ID NO: 13 - DNA, 18 bp, Sus scrofa
  misc_feature (4)..(4): n is a, c, g, or t
  misc_feature (13)..(13): n is a, c, g, or t
cggngaccgcctnatcag

SEQ ID NO: 14 - DNA, 20 bp, Sus scrofa
tcagtgycagtcraatgttc

SEQ ID NO: 15 - DNA, 27 bp, Sus scrofa
attctagaggccgaggcggccgacatg

SEQ ID NO: 16 - DNA, 19 bp, Sus scrofa
gcacccccagatcgacccc

SEQ ID NO: 17 - DNA, 23 bp, Sus scrofa
caaactcctcaataaactgcacg

SEQ ID NO: 18 - DNA, 48 bp, Sus scrofa
ggggacaagtttgtacaaaaaagcaggctcagcatggccgcgctgctc

SEQ ID NO: 19 - DNA, 52 bp, Sus scrofa
gggaccactttgtacaagaaagctgggtttagtgccagtcaaatgttctgcc

SEQ ID NO: 20 - DNA, 19 bp, Sus scrofa
agatgactggtcgggctgc

SEQ ID NO: 21 - DNA, 23 bp, Sus scrofa
caatgatrtggctcatgtagtcc

SEQ ID NO: 22 - DNA, 25 bp, Sus scrofa
atggttgagcgcgccagcaagttcg

SEQ ID NO: 23 - DNA, 24 bp, Sus scrofa
ggttattggccaggttgtaggggc

SEQ ID NO: 24 - DNA, 28 bp, Sus scrofa
attctagaggccgaggcggccgacatgt

SEQ ID NO: 25 - DNA, 18 bp, Sus scrofa
ggacctcttccagcagcg

SEQ ID NO: 26 - DNA, 21 bp, Sus scrofa
gctatcagtacaagcggcagc

SEQ ID NO: 27 - DNA, 16 bp, Sus scrofa
ccaggctcagccccgg

SEQ ID NO: 28 - DNA, 39 bp, Sus scrofa; identical to SEQ ID NO: 8

SEQ ID NO: 29 - DNA, 23 bp, Sus scrofa
ggcaatgtcgacctccctacaac

SEQ ID NO: 30 - DNA, 17 bp, Sus scrofa
tcagccccgggcccgcg

SEQ ID NO: 31 - DNA, 23 bp, Sus scrofa
ctccctacaacccgaattcctac

SEQ ID NO: 32 - DNA, 19 bp, Sus scrofa
gcccgcgtactggtagagg

SEQ ID NO: 33 - DNA, 56 bp, Sus scrofa
ggggacaagtttgtacaaaaaagcaggcttaggacaatggtgacacatgcggcggc

SEQ ID NO: 34 - DNA, 55 bp, Sus scrofa
ggggaccactttgtacaagaaagctgggtcctaccacttctcgatgatgtggctc

(19) United States
(12) Patent Application Publication
(10) Pub. No.: US 2008/0064095 A1
(43) Pub. Date: Mar. 13, 2008

(54) METHOD FOR OBTAINING MASTOCYTE LINES FROM PIG TISSUES AND FOR PRODUCING HEPARIN-TYPE MODULES

(75) Inventors: Jean-Marc GUILLAUME, Paris (FR); Werner DITTRICH, Frankfurt (DE); Sandrine PEREZ, Alfortville (FR); Christine Michelle Pierrette ANDREONI, Villete D'Anthone (FR); Romain PAILLOT, Lyon (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: AVENTIS PHARMA S.A., Antony Cedex (FR)

(21) Appl. No.: 11/938,422

(22) Filed: Nov. 12, 2007

Related U.S. Application Data
(62) Division of application No. 10/823,142, filed on Apr. 13, 2004.
(60) Provisional application No. 60/477,962, filed on Jun. 12, 2003.

(30) Foreign Application Priority Data
Apr. 14, 2003 (FR) 0304671

(51) Int. Cl. C12N 5/00

(52) U.S. Cl. 435/325

(57) ABSTRACT

The present application relates to a method for obtaining mastocyte cultures or lines. It also relates to a method for producing heparin-type molecules, comprising the culturing of mastocyte cultures or lines.

Mastocytes are cells of the immune system, derived from hematopoietic precursors, which are involved in the inflammatory response, in particular in the phenomena of allergy and hypersensitivity. They are located in the connective tissue, in particular in the skin, the intestinal mucosa and the respiratory mucosa.

Mastocytes have the appearance of rounded cells with a diameter of between approximately 5 and 25 μm, and have a single, central or off-center, rounded nucleus. They are also characterized by the presence of many metachromatic cytoplasmic granulations.

These granules contain various molecular species which have pro-inflammatory activity, such as histamine, serotonin, proteoglycans such as heparin or chondroitin sulfate, enzymes, cytokines, and eosinophil- and neutrophil-chemoattracting factors. These species are released during mastocyte activation.

After activation, a secondary response is initiated, during which the synthesis of mediators occurs, such as leucotrienes, prostaglandins, PAF (platelet activating factor), interleukins (IL-4, IL5, IL-6, IL10, IL12 and IL 13), cytokines (TGF beta, gamma IFN, GM-CSF) and chimiokines (MCP-1, IL8, RANTES). All of these species contribute to the triggering of an inflammatory process and to the setting up of a T lymphocyte-dependent specific immune response.

Mastocyte cultures have already been obtained in humans and in mice, but the state of the art provides no description of such established cultures or of lines in pigs. Razin et al (J. Biol. Chem., 257, 7229-7236, 1982) describe the obtaining of mouse mastocytes using culture media containing IL-3. Wang et al (Circ. Res., 84, 74-83, 1999) describe the isolation of serum mastocytes obtained from rat pleural and peritoneal cavities. Various molecular species are produced, but only when the mastocytes are cocultured with rat aorta smooth muscle cells. Application WO99/26983 describes very similar studies, and is relatively imprecise regarding the application to other species.

Cell lines have been established in mice (Montgomery et al, Proc. Natl. Acad. Sci. USA, 89, 11327-11331, 1992 and Application WO90/14418), but from mastocytomas. These tumors are extremely rare in pigs.

In humans, obtaining mastocyte cultures has proved to be difficult. It was first of all possible using a system of coculturing with fibroblasts (Ishizaka et al, Current Opinion in Immunology, 5, 937-943, 1993). Other authors then succeeded in obtaining mastocytes from intestinal cells and in maintaining these cells in culture for approximately 6 months in the presence of SCF (Bischoff et al, J. Immunol., 159, 5560-5567, 1997). When it was measured, the authors of these various articles reported only a small production of heparin-type compounds.

In pigs, Emery et al (Experimental Hematology, 24, 927-935, 1996) have maintained cultures of cells obtained from bone marrow, for 7 weeks. However, it appears that the cultures obtained are mixtures of various cell types and not homogeneous cultures or lines of mastocytes. In addition, these cultures contain cells which are undifferentiated in homogeneous cultures of mastocytes. Ashraf et al (Veterinary Parasitology; 29, 134-158, 1988) have isolated pig mastocytes from the intestinal mucosa, without maintaining amplifiable cultures. In addition, characterization of the isolated mastocytes reveals an absence of heparin.

Heparin belongs to the glycosaminoglycan (GAG) family, which includes linear polysaccharides containing a repeat of a disaccharide sequence made up of an amino sugar (D-glucosamine or galactosamine) and a uronic acid (D-glucuronic or L-iduronic acid).

In the case of heparin, which belongs, with heparin sulfate, to the glucose aminoglycan subfamily, the amino sugar is D-glucosamine. The uronic acid is either glucuronic acid (Glc) or iduronic acid (Ido). The glucosamine can be N-acetylated, N-sulfated and O-sulfated.

Conventionally, the term “heparin” denotes highly sulfated polysaccharides in which more than 80% of the glucosamine residues are N-sulfated and the number of O-sulfates is greater than that of the N-sulfates. The sulfate/carboxylate ratio is generally greater than 2 for heparin. However, the structure of heparin is in fact very heterogeneous, and chains exist which may contain very different ratios. Like all GAGs, heparin is synthesized in the form of a proteoglycan essentially by mastocytes.

The first step in heparin synthesis is the formation of the ser-glycine protein core, consisting of repeating serine and glycine residues. Elongation of the heparin chain occurs by addition of a tetrasaccharide, and then by successive additions of glucosamines and of uronic acids regularly alternated.

The proteoglycan thus formed undergoes many sequential transformations: N-deacetylation, N-sulfation, D-glucuronic acid epimerization, and O-sulfation. However, this complete maturation only takes place on part of the proteoglycan, which generates a great structural variability of the heparin, responsible for its heterogeneity.

The polysaccharide chains are then cleaved from the ser-glycine by an endoglucuronidase. These chains then have a molecular weight of between 5,000 and 30,000 Da. They form complexes with basic proteases and are thus stored in the mastocyte granules. The heparin is excreted only during mastocyte degranulation.

Heparin plays an important biological role, in particular in hemostases, and is very widely used in therapeutics, in particular as an anticoagulant and an antithrombotic agent.

Most of the heparin used is isolated from pig intestinal mucosa, from where it is extracted by proteolysis, followed by purification on anion exchange resin (for a review of the various methods for preparing heparin, cf. DUCLOS, “L'Héparine: fabrication, structure, propriétés, analyse”; [Heparin: production, structure, properties, analysis]; Ed. Masson, Paris, 1984).

Analysis of the disaccharide composition of pig heparin after depolymerization and chromatography makes it possible to differentiate heparin from the other glycosaminoglycans. Eight main disaccharides are in particular distinguished (FIG. 6). The sulfated disaccharides, Is, IIs, IIs, IVs, are proportionally the most abundant, with the major one being Is, the amount of which is greater than 40%, and preferably greater than 50%. The order of abundance is then the disaccharides IIs, IIIs and IVs. The ratio between the Is and IIs disaccharides is between 3 and 8.

A heterogeneity may be observed in the composition of the heparin between batches derived from batches of animals of different origins. This heterogeneity is liable to engender variabilities in biological activity.

In addition, the use of animals as a source of heparin constitutes a risk due to the possible presence of viruses able to be transmitted to humans.

In addition, the supply of raw material may prove to be irregular.

The present invention proposes to overcome these drawbacks and to avoid the problems of supply in terms of quantity and quality, using a source of raw material which is more readily controllable.

The Applicant has shown that it is possible to produce, in considerable amounts and from mastocyte cultures, heparin having properties which are comparable to those of heparin extracted from porcine intestinal mucus and which are reproducible.

The Applicant has also demonstrated that the genes encoding three proteins which are important for the production of heparin-type molecules or the independence of mastocytes with respect to growth factors exhibit, in pigs, sequences different from those of other species.

A subject of the present invention is a method for obtaining mastocyte cultures or lines, comprising the culturing of a population of bone marrow stem cells from young pigs or from fetuses, in a medium comprising at least approximately 0.2 ng/ml of preferentially porcine interleukin-3 (IL-3) (preferentially at least 0.5 ng/ml, even more preferentially at least 2 ng/ml), at least approximately 8 ng/ml of preferentially porcine Stem Cell Factor (SCF), (preferentially at least 20 ng/ml, even more preferentially at least 80 ng/ml) and at least approximately 0.1 ng/ml of preferentially porcine interleukin-4 (IL-4) (preferentially at least 0.5 ng/ml, even more preferentially at least 1 ng/ml), 10 ng/ml of preferentially porcine interleukin-6 (IL-6) (preferentially at least 50 ng/ml, even more preferentially at least 100 ng/ml) and/or 1 ng/ml of preferentially porcine G-CSF (preferentially at least 5 ng/ml, even more preferentially at least 10 ng/ml).

Thus, the production medium contains a combination of IL-4, IL-6 and G-CSF, separately, in pairs, or all three, in a medium containing IL-3 and SCF.

Although these various factors are preferentially of porcine origin, that is to say their sequence is deduced from that of the corresponding factor in pigs, it is possible to replace at least one of them with a factor of another origin. The interleukin 4 (IL-4), although preferentially of porcine origin, may also be of murine or human origin.

According to one embodiment of this method, the pigs from which the stem cells are derived are between approximately 2 days old and approximately 6 weeks old. However, the method may be applied to cells derived from embryos or from older pigs.

Advantageously, the cells are maintained in the medium for at least approximately 30 days.

A subject of the present invention is also porcine mastocyte cultures and lines which can be obtained using said method.

The term “mastocytes” is intended to mean cells which, among other characteristics, exhibit metachromatic cytoplastic granules containing heparin-type molecules and proteases such as tryptase, and express at their surfaces receptors such as the SCF receptor, known as c-kit, or else the IgE receptor.

The term “culture” denotes herein, generally, a cell or a set of cells cultured in vitro. A culture developed directly from a cell or tissue sample taken from an animal is referred to as a “primary culture”.

The term “line” is used when at least one passage and generally several consecutive passages, of subculturing have been successfully carried out, and denotes any culture which is derived therefrom (SCHAEFFER, In Vitro Cellular and Developmental Biology, 26, 91-101, 1990).

A subject of the present invention is furthermore porcine mastocyte cultures or lines, which produce heparin-type molecules exhibiting a ratio between the IIs and IIIs disaccharides close to that of porcine heparin.

The expression “heparin-type molecules” is intended to mean highly sulfated polysaccharides in which more than 80% of the glucosamine residues are N-sulfated and the number of O-sulfates is greater than that of the N-sulfates.

Advantageously, such cultures or lines produce heparin-type molecules exhibiting a ratio between the IIs and IIIs disaccharides of between 0.5 and 5 (preferentially between 1 and 2.5, even more preferentially between 1.3 and 1.9) and/or heparin-type molecules exhibiting a ratio between the Is and IIs disaccharides of between approximately 3 and 8 (preferentially between 4 and 7, even more preferentially between 5 and 7).

Established cultures or lines of porcine mastocytes according to the present invention can also produce at least 0.1 μg of heparin-type molecules/10⁶ cells (preferentially at least 1 μg, even more preferentially at least 10 μg).

Advantageously, such cultures or lines produce heparin-type molecules in which the amounts of Is disaccharides are greater than the amounts of IIs disaccharides, the amounts of IIs disaccharides are greater than the amounts of IIIs disaccharides, and the amounts of IIIs disaccharides are greater than the amounts of IVs disaccharides.

According to another advantageous embodiment, such cultures or lines produce heparin-type molecules exhibiting ratios between the Is, IIs, IIIs and IVs disaccharides close to those of heparin.

Advantageously, such cultures or lines produce heparin-type molecules comprising at least 30% of Is disaccharides (preferentially at least 40%, even more preferentially at least 50%).

Advantageously, such cultures or lines produce heparin-type molecules exhibiting an anti-Xa activity greater than at least 10 IU/mg (preferentially at least 20 IU/mg) and/or exhibiting an anti-IIa activity greater than at least 10 IU/mg (preferentially at least 20 IU/mg).

According to a preferential embodiment of the invention, such lines are the porcine mastocyte lines deposited with the Collection de Cultures de Microorganisms (The Collection of Cultures and Mimmcroorganisms] of the Institut Pasteur (CNCM) 28 rue du Docteur Roux, 75724 Paris cedex 15, France, on Apr. 9, 2003, respectively under the numbers I-3010, I-3011, I-3012, I-3013, I-3014.

These lines, deposited with the CNCM, have the advantage of having been obtained from pigs satisfying health requirements consistent with use of the products derived from their cells in human therapeutics. These pigs are derived from protected, pig specific pathogen free (SPF) colonies.

Nucleic acids comprising genes encoding factors capable of improving the characteristics of the cultures and lines according to the present invention may be introduced into these cells. The term “nucleic acid” is used to denote a DNA or an RNA. Advantageously, it is a complementary or genomic DNA.

Such factors can make it possible either to promote the growth of the cells or to modulate the composition of the biological molecules which they produce, and in particular the composition of the heparin-type molecules.

They may be genes encoding immortalizing proteins, such as the simian virus 40 (SV40) T antigen, the E6 and E7 proteins of the human papilloma virus HPV, the E1A proteins of the adenovirus, the EBNA2 proteins of the Epstein-Barr virus or else the Tax proteins of the HTLV-1 virus. The nucleic acid encoding the catalytic subunit of telomerase, TERT, can also be used as immortalizing gene.

The SV-40 virus AgT will preferentially be used; the sequence of the complementary DNA of this antigen is available in GenBank under the reference NC_{—}001669.

They may also be genes encoding proteins which allow the cells to proliferate, such as, for example, G-CSF, SCF and interleukins (IL-3, IL-4 and IL-6).

Recently, the study of murine and human mastocytomas has made it possible to identify mutations or deletions of the c-kit gene, responsible for constitutive activation of the c-kit receptor. Expression of the c-kit gene mutated at V814, in IC2 immature mastocytes, induces transformation of these cells, namely the acquiring of SCF-independent growth and of a tumorigenic potential (Pia et al, Blood, 87(8), 3117-3123, 1996). A nucleic acid comprising such a mutated gene can be introduced into these cells.

They may be genes encoding proteins such as ser/gly or enzymes which act on the sulfation of the heparin-type molecules. Such an enzyme may be an O-sulfatase, such as a 3-O-sulfatase, or else a 6-O-sulfatase. Advantageously, such an enzyme is 3 O-sulfatase-1 (3-OST-1), preferentially porcine 3-O-sulfatase-1.

The nucleic acids comprising these genes can be introduced into these cells by any method known to those skilled in the art, and in particular by transfection, by nucleoporation or by electroporation. Retroviral vectors carrying these genes can also be used to transfect these cells.

In the context of the present invention, the applicant has demonstrated that the introduction of a nucleic acid encoding a 3-OST, and in particular 3-OST-1, makes it possible to modulate the composition of the heparin-type molecules of the mastocytes, whatever the type of mastocyte of porcine origin.

The Applicant does not therefore intend to limit this subject of its invention to mastocytes obtained using the method described above. Thus, a subject of the present application is any mastocyte of porcine origin, into which a nucleic acid encoding a 3-OST has been introduced.

The Applicant has also determined the sequences of the three proteins of porcine origin which can be used to implement the present invention and nucleotide sequences encoding these proteins.

Thus, a subject of the present application is a protein of porcine origin of the c-kit type, which has a C-terminal end having the sequence SEQ ID NO. 3. Such a protein can comprise a sequence exhibiting at least 99% identity with the sequence SEQ ID NO. 2. Preferentially, such a protein has a glutamine (Q) at position 40 and/or a lysine (K) at position 173. A subject of the present invention is also a polynucleotide or a nucleic acid comprising a sequence encoding a protein of porcine origin of the c-kit type. Such a nucleic acid can comprise a sequence exhibiting at least 99% identity with the sequence SEQ ID NO. 1.

The obtaining of the complete sequence of the porcine c-kit was not evident in view of the state of the art.

The subject of the present invention is also a protein of porcine origin exhibiting 3-O-sulfatase activity. Such a protein can comprise a sequence exhibiting at least 95%, preferentially at least 97%, and even more preferably at least 99%, amino acid identity with a protein of sequence SEQ ID NO. 5.

A subject of the present invention is also a polynucleotide or a nucleic acid comprising a sequence encoding a protein of porcine origin exhibiting 3-OST activity. Such a nucleic acid can comprise a sequence exhibiting at least 95%, preferentially at least 97%, and even more preferentially at least 99%, nucleotide identity with a nucleic acid of sequence SEQ ID NO. 4.

The obtaining of the sequence of the porcine 3-OST was not evident in view of the state of the art. The isolated porcine 3-OST is liable to exhibit unexpected properties, and in particular to exhibit better activity in the porcine mastocytes compared to the 3-OSTs of other species known to those skilled in the art.

A subject of the present application is also a protein of porcine origin exhibiting 6-O-sulfatase activity. Such a protein can comprise a sequence exhibiting at least 90%, preferentially at least 95%, and even more preferentially at least 99%, amino acid identity with a protein of sequence SEQ ID NO. 7. A subject of the present invention is also a polynucleotide or a nucleic acid comprising a sequence encoding a protein of porcine origin exhibiting 6-OST activity. Such a nucleic acid can comprise a sequence exhibiting at least 95%, preferentially at least 97%, and even more preferentially at least 99%, nucleotide identity with a nucleic acid of sequence SEQ ID NO. 6.

The obtaining of the sequence of the porcine 6-OST was not evident in view of the state of the art. The isolated porcine 6-OST is liable to exhibit unexpected properties, and in particular to exhibit better activity in the porcine mastocytes compared to the 3-OSTs of other species known to those skilled in the art.

In addition a subject of the present application is nucleic acids which hybridize, under high stringency conditions, with a nucleic acid of sequence SEQ ID NO. 1, SEQ ID NO. 4 or SEQ ID NO. 6.

For the purpose of the present invention, the “percentage identity” between two nucleotide or amino acid sequences can be determined by comparing two optimally aligned sequences through a window of comparison. The part of the nucleotide or polypeptide sequence in the window of comparison may thus comprise additions or deletions (for example gaps) compared to the reference sequence (which does not comprise these additions or deletions) so as to obtain optimal alignment of the two sequences.

The percentage is calculated by determining the number of positions at which an identical nucleic acid base or amino acid residue is observed for the two (nucleic acid or peptide) sequences compared, and dividing the number of positions at which there is identity between the two bases or amino acid residues by the total number of positions in the window of comparison, and then multiplying the result by 100 in order to obtain the percentage sequence identity.

The optimal alignment of the sequences for the comparison can be carried out on a computer using known algorithms contained in the WISCONSIN GENETICS SOFTWARE PACKAGE, GENETICS COMPUTER GROUP (GCG), 575 Science Drive, Madison, Wis.

By way of illustration, the percentage sequence identity may be effected using the BLAST software (versions BLAST 1.4.9 of March 1996, BLAST 2.0.4 of February 1998 and BLAST 2.0.6 of September 1998), using exclusively the default parameters (S. F Altschul et al, J. Mol. Biol. 1990 215: 403-410, S. F Altschul et al, Nucleic Acids Res. 1997 25: 3389-3402). Blast searches for sequences similar/homologous to a reference “request” sequence, using the algorithm of Altschul et al. The request sequence and the data bases used may be peptide-based or nucleic acid-based, any combination being possible.

For the purposes of the present invention, the expression “high stringency hybridization conditions” will be intended to mean the following conditions:
1—Prehybridization of the membranes and:
   Mix: 40 μl of salmon sperm DNA (10 mg/ml)+40 μl of human placental DNA (10 mg/ml).
   Denature for 5 min at 96° C., and then immerse the mixture in ice.
   Remove the 2×SSC and pour 4 ml of formamide mix into the hybridization tube containing the membrane.
   Add the mixture of the two denatured DNAs.
   Incubate at 42° C. for 5 to 6 hours, with rotation.
2—Labeled probe competition:
   Add to the labeled and purified probe 10 to 50 μl of Cot I DNA, depending on the amount of repetitions.
   Denature for 7 to 10 min at 95° C.
   Incubate at 65° C. for 2 to 5 hours.
3—Hybridization:
   Remove the prehybridization mix.
   Mix 40 μl of salmon sperm DNA+40 μl of human placental DNA; denature for 5 min at 96° C., and then immerse in ice.
   Add to the hybridization tube 4 ml of formamide mixture, the mixture of the two DNAs and the denatured labeled probe/Cot I DNA.
   Incubate for 15 to 20 hours at 42° C., with rotation.
4—Washing:
   One wash at ambient temperature in 2×SSC, to rinse.
   2 times 5 minutes at ambient temperature, 2×SSC and 0.1% SDS at 65° C.
   2 times 15 minutes at 65° C., 1×SSC and 0.1% SDS at 65° C.
Wrap the membranes in Saran wrap and expose.

The hybridization conditions described above are suitable for the hybridization, under high stringency conditions, of a nucleic acid molecule of length varying from 20 nucleotides to several hundred nucleotides.

It goes without saying that the hybridization conditions described above can be adjusted as a function of the length of the nucleic acid the hybridization of which is desired, or of the type of labeling chosen, according to techniques known to those skilled in the art.

The suitable hybridization conditions may, for example, be adjusted according to the teachings contained in the work by HAMES et HIGGINS (1985, “Nucleic acid hybridization: a practical approach”, Hames and Higgins Ed., IRL Press, Oxford) or else in the work by F. AUSUBEL et al (1989, Current Protocols in Molecular Biology, Green Publishing Associates and Wiley Interscience, N.Y).

The proteins which are a subject of the present invention can be obtained by any means known to those skilled in the art. They are, however, advantageously obtained by expression of the nucleic acids as described above, encoding these proteins, optionally inserted into expression vectors, in cells advantageously chosen, optionally followed by an extraction and a purification which may be total or partial.

The invention also relates to a recombinant vector comprising a nucleic acid according to the invention.

Advantageously, such a recombinant vector will comprise a nucleic acid chosen from the following nucleic acids:
a) a nucleic acid encoding a protein having at least 60% amino acid identity with a sequence SEQ ID NO. 5 or SEQ ID NO. 7 or a peptide fragment or a variant thereof;
b) a nucleic acid comprising a nucleic acid having a sequence of SEQ ID NO. 1, SEQ ID NO. 4 or SEQ ID NO. 6, or a fragment or a variant thereof;
c) a nucleic acid having at least 60% nucleotide identity with a nucleic acid having a sequence SEQ ID NO. 4 or SEQ ID NO. 6, or a fragment or a variant thereof;
d) a nucleic acid which hybridizes, under high stringency hybridization conditions, with a nucleic acid of sequence SEQ ID NO. 1, SEQ ID NO. 4 or SEQ ID NO. 6, or a fragment or a variant thereof.

For the purpose of the present invention, the term “vector” will be intended to mean a circular or linear, DNA or RNA molecule which may equally be in single-stranded or double-stranded form.

According to one embodiment, the expression vector comprises, besides a nucleic acid in accordance with the invention, regulatory sequences for directing the transcription and/or the translation thereof.

According to an advantageous embodiment, a recombinant vector according to the invention will in particular comprise the following elements:
(1) elements for regulating the expression of the nucleic acid to be inserted, such as promoters and enhancers;
(2) the coding sequence included in the nucleic acid in accordance with the invention to be inserted into such a vector, said coding sequence being placed in phase with the regulatory signals described in (1); and
(3) suitable transcription initiation and stop sequences.

In addition, the recombinant vectors according to the invention may include one or more origins of replication in the cellular hosts in which their amplification or their expression is desired, markers or selection markers.

Cells comprising such nucleic acids and/or expressing such proteins constitute other subjects of the present invention. The present application also relates to a method for producing heparin-type molecules, comprising the culturing of porcine mastocyte cultures or lines as described above.

The mastocytes, obtained according to the invention in a medium containing IL-3, SCF and IL-4, exhibit a disaccharide structure which is better than those obtained in the medium containing only IL-3 and SCF.

The Applicant has also shown that the addition of IL-4 to the culture medium makes it possible to obtain, from the mastocytes, heparin-type molecules exhibiting characteristics which are closer to porcine heparin compared to those obtained using cells obtained in a medium containing only IL-3 and SCF or containing IL-3, SCF and IL-6 or IL-3, SCF and G-CSF.

Thus, the present application also relates to a method for producing heparin-type molecules, comprising the culturing, in a suitable medium, of porcine mastocyte cultures or lines in a culture medium comprising at least approximately 0.1 ng/ml of IL-4 (preferentially at least approximately 0.5 ng/ml, even more preferentially at least approximately 1 ng/ml).

Mastocytes can also be modified in order to overexpress IL-4. Thus, another subject of the present application is a method for producing heparin-type molecules, comprising the obtaining of porcine mastocyte cultures or lines transfected with a nucleic acid encoding IL-4, and the culturing of these cells in a suitable culture medium. Such mastocytes constitute, in themselves, a subject of the present application.

They can be obtained by any method known to those skilled in the art, and in particular by transfection, by nucleoporation or by electroporation of a nucleic acid comprising a gene encoding IL-4. Retroviral vectors carrying these genes can also be used to transfect the cells. The sequence of the complementary DNA of IL-4 was described by Bailey et al (Biotic. Biophys. Acta. 1171(3), 328-330, 1993).

The cells, lines and cultures according to the present invention can be maintained in culture under the conditions under which they were obtained. They can also be maintained in culture in media comprising decreased amounts of SCF, GM-CSF, IL-3, IL-4 and/or IL-6. They will however preferentially be maintained in a medium containing IL-4.

These mastocytes will preferably be cultured in a defined culture medium ((MEMα/DMEM, RPMI, IMDM, . . . ) supplemented with growth factors, used in combination or individually.

The media may also be supplemented with bovine serum, at a concentration of between 0.5% and 20% (v/v).

The addition of bovine serum to the culture media can be replaced with the use of a serum-free culture medium such as AIMV (INVITROGEN) so as to reduce the protein concentration of the medium and the risks associated with the use of compounds of animal origin (KAMBE et al., J. Immunol. Methods, 240, 101-10, 2000).

The independent nature of the cells, with respect to the addition of serum and/or to the use of growth factors, can be obtained by mutation of the cell phenotype through the action of transforming and/or immortalizing agents (TSUJIMURA, Pathology International, 46, 933-8, 1996; PIAO and BERNSTEIN, Blood, 87(8), 3117-23, 1996).

The mastocytes can be cultured using the techniques developed for the bulk culture of eucaryotic cells, as described for example, by GRIFFITHS et al. (Animal Cell Biology, Eds. Spier and Griffiths, Academic Press, London, vol. 3, 179-220, 1986). Use may be made of bioreactors with a volume greater than m³, as described by PHILIPS et al. (Large Scale Mammalian Cell Culture, Eds. Feder and Tolbert, Academic Press, Orlando, U.S.A., 1985), or by MIZRAHI (Process Biochem, Aug. 9-12, 1983).

The culturing may also be carried out in a suspension or on a microsupport according to the technique described by VAN WEZEL (Nature, 216, 64-65, 1967).

Use may also be made of batch culture systems, which are commonly used for eucaryotic cell cultures, due to the fact that they are very much simpler to use on an industrial scale ((VOGEL and TODARO, Fermentation and Biochemical Engineering Handbook, 2^{nd} edition, Noyes Publication, Westwood, N.J., U.S.A., 1997). The cell densities obtained with these systems are generally between 10⁶ and 5×10⁶ cells/ml.

The productivity of the batch cultures can advantageously be increased by removing a portion of the cells from the bioreactor (70% to 90%) for the GAG extraction and heparin isolation operations, and keeping the remaining cells in the same bioreactor in order to initiate a new culture. In this method of culturing, referred to as repeat-batch culture, it is also possible to distinguish the optimum parameters for the cell growth phase from those allowing greater accumulation of GAGS and of heparin within the cells.

Perfusion-fed continuous culture systems, with or without cell retention, can also be used (VELEZ et al., J. Immunol. Methods, 102(2), 275-278, 1987; CHAUBARD et al., Gen. Eng. News, 20, 18-48, 2000).

In the context of the present invention, use may particularly be made of perfusion-fed culture systems which allow retention of the cells inside the reactor, and result in growth and production greater than that which can be obtained by batch. The retention may be effected via retaining systems of the spin-filter, hollow fiber or solid matrix type (WANG et al., Cytotechnology, 9, 41-49, 1992; VELEZ et al., J. Immunol. Methods, 102(2), 275-278, 1987)

The cell densities obtained are generally between 10⁷ and 5×10⁷ cells/ml. Culturing in bioreactors allows, by using on-line measuring sensors, better control of the physicochemical parameters of the cell growth: pH, pO₂, Red/Ox, growth substrates such as vitamins, amino acids or carbon-containing substrates (for example glucose, fructose, galactose), metabolites such as lactate or aqueous ammonia, etc.

It may be envisioned to quantitatively and qualitatively increase the content of heparin-type molecules of the mastocytes subsequent to treatment with sodium butyrate (Nakamura and al., Biochim. Biophys. Acta. 627, 60-70, 1980).

After culturing for 3 to 14 days, preferably after 3 to 5 days, the cells are harvested and separated from the culture medium, generally by centrifugation or filtration.

Various centrifugation systems can be used; mention will, for example, be made of those described by VOGEL and TODARO (Fermentation and Biochemical Engineering Handbook, 2^{nd} Edition, Noyes Publication, Westwood, N.J., U.S.A.).

Alternatively to or in combination with the centrifugation, the separation may be carried out by tangential microfiltration using membranes with a porosity of less than the average diameter of the cells (5 to 20 μm) while at the same time allowing the other compounds in solution/suspension to pass. The tangential flow rate and the pressure applied to the membrane will be chosen so as to generate little shear force (number of Reynolds less than 5000 sec⁻¹) in order to reduce clogging of the membranes and to preserve the integrity of the cells during the separation operation.

Various membranes can be used; for example, spiral membranes (AMICON, MILLIPORE), flat membranes or hollow fibers (AMICON, MILLIPORE, SARTORIUS, PALL, GF).

It is also possible to choose membranes for which the porosity, the charge or the grafting make it possible to perform a separation and a first purification with respect to possible contaminants which may be present in the culture medium, such as cell proteins, DNA, viruses or other macromolecules.

The use of membranes with a smaller porosity can also be envisioned when heparin has been released from the intracellular content, by degranulation or lysis of some of the mastocytes, and is present in the culture medium at the time of the separation step. In this case, the cell separation is combined with an ultrafiltration step over one or more membranes which have a porosity and are arranged such that it is possible to concentrate the heparin and to separate it from the other species present in the medium, as a function of the size of the molecular weight, and optionally of the electrical charge or of the biological properties.

In the context of this embodiment, the cut-off threshold of the membranes is preferably between 1000 and 5 Kda. Use may be made of membrane systems similar to those used for microfiltration, for example spiral membranes, flat membranes or hollow fibers. Use may advantageously be made of membranes which make it possible to separate and purify the heparin, due to their charge properties or to the grafting of ligands exhibiting affinity for heparin (for example antibodies, ATIII, lectin).

However, use will in general preferably be made of methods for producing and harvesting cells which make it possible to keep the heparin in the intracellular content.

The recovery of the heparin from the mastocytes can also be carried out after degranulation or lysis of the cells.

The degranulation can be brought about by the binding of specific ligands to the receptors present at the surface of the mastocytes, for example the binding of agents of the allergen type (such as IgE Fc fragment or analogs of this fragment) to the IgE receptors of the mastocytes.

Other agents can also induce mastocyte degranulation. These agents can be classified in several categories, such as cytotoxic agents, enzymes, polysaccharides, lectins, anaphylatoxins, basic compounds (compound 48/80, substance P, etc.) or calcium (ionophore A23187, ionomycin, etc.) [D. Lagunoff and T. W. Martin. 1983. Agents that release histamine from mast cells. Ann. Rev. Pharmacol. Toxicol., 23:331-51]. A degranulating agent can be used repeatedly on the same cells maintained in culture. In this method of production, the productivity is significantly increased by the simplification of the method of harvesting from the supernatant and by the maintaining of the cells in culture.

In the particular case of the ionophore A23187, the mastocyte degranulation can be induced, for example, by treating 2.10⁶ cells/ml of mastocytes with the ionophore A23187 at concentrations of between 1 and 100 μg/ml and for periods of action ranging from 1 minute to 4 hours.

Mastocyte lysis can be induced, for example, by osmotic shock using hypotonic or hypertonic solutions, by thermal shock (freezing/thawing), by mechanical shock (for example sonication or pressure variation), by the action of chemical agents (NaOH, THESIT™, NP40™, TWEEN 20™, BRIJ-58™, TRITON X™-100, etc.) or by enzyme lysis (papain, trypsin, etc.), or by a combination of two or more of these methods.

To extract and purify the heparin from the cell lysate, to separate the polysaccharide chains from the ser-glycine core, and to separate the heparin chains from the other GAGs present in the extraction medium, use may be made of methods similar to those used in the context of the extraction and purification of heparin from animal tissues, which are known in themselves, and described in general works, such as the manual by DUCLOS, mentioned above.

By way of nonlimiting examples, to separate the heparin from the nucleic acids and from the cell proteins, and to solubilize it, i.e. to break the bonds with the ser-glycine core:
the cell lysate can be subjected to one or more enzyme digestions (pronase, trypsin, papain, etc.);
the heparin-protein bonds can be hydrolyzed in alkaline medium, in the presence of sulfates or of chlorides;
treatment in acid medium (for example with trichloracetic acid under cold conditions) can also be carried out in order to destroy the nucleic acids and the proteins originating from the cells, supplemented by the use of an ionic solution which makes it possible to dissociate the GAG-protein interactions.

It is also possible to perform an extraction with guanidine, after enzyme hydrolysis; in order to purify the solubilized heparin, it can, for example, be precipitated with potassium acetate, with a quaternary ammonium, with acetone, etc.

These purification steps can advantageously be supplemented or replaced with one or more chromatography steps, in particular anion exchange chromatography steps.

The subject of the present invention is also the heparin preparations which can be obtained from mastocyte cultures using a method according to the invention.

The heparin preparations in accordance with the invention, which have biological properties comparable to those of the heparin preparations obtained in the prior art from animal tissues, can be used in all the usual applications of heparin.

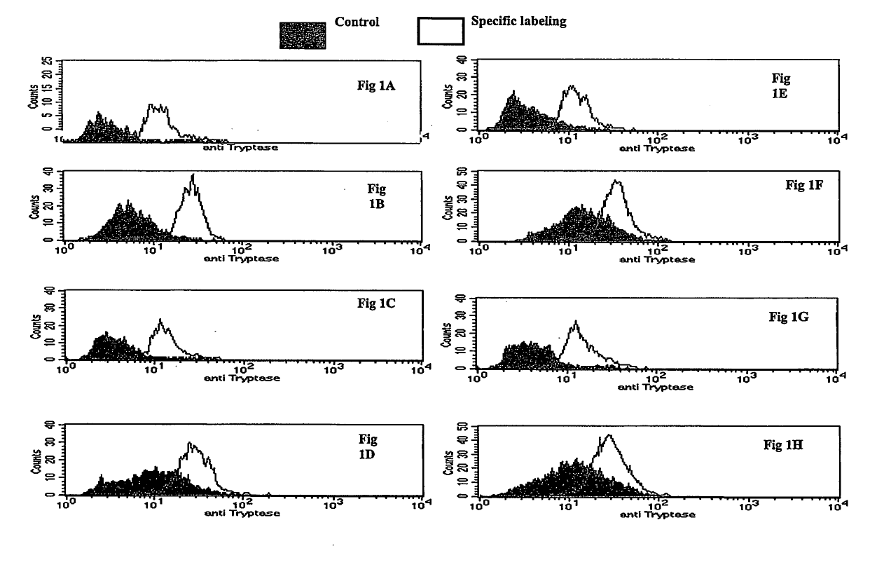

FIGS. 1A to 1H illustrate the anti-tryptase labeling of mastocytes obtained after culturing for 3 weeks, respectively under the conditions C1 to C8. The dark and light peaks correspond, respectively, to the controls (without antibody) and to the cells obtained under the conditions C1 to C8.

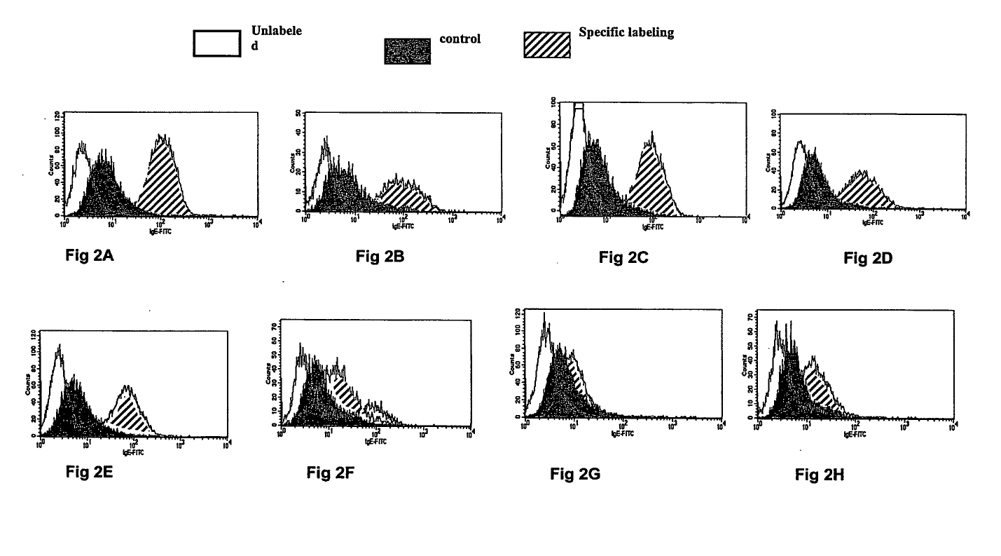

FIGS. 2A to 2H illustrate the labeling of the IgE receptors of the mastocytes obtained after culturing for 5 weeks, respectively under the conditions C1 á C8. The hatched, dark and light peaks correspond, respectively, to unlabeled cells (non-mastocytic porcine cells), to control cells and to the cells obtained under the conditions C1 to C8.

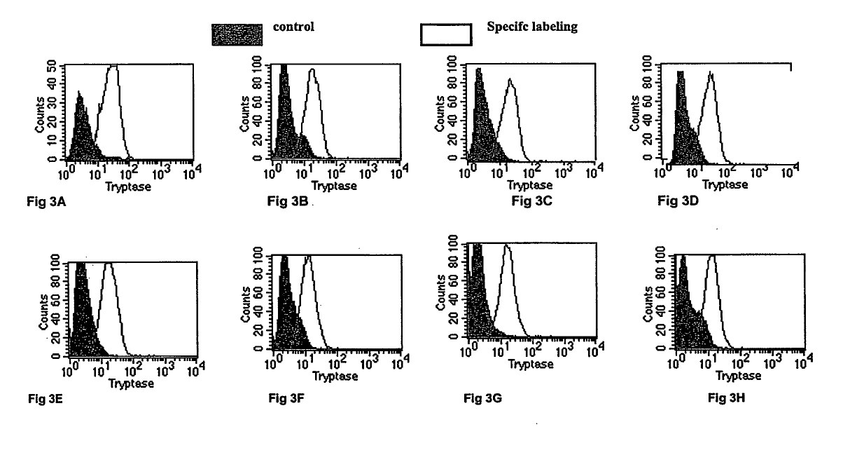

FIGS. 3A to 3H illustrate the anti-tryptase labeling of mastocytes obtained after culturing for 7 weeks, respectively under the conditions C1 to C8. The dark and light peaks correspond, respectively, to the controls and to the cells obtained under conditions C1 to C8.

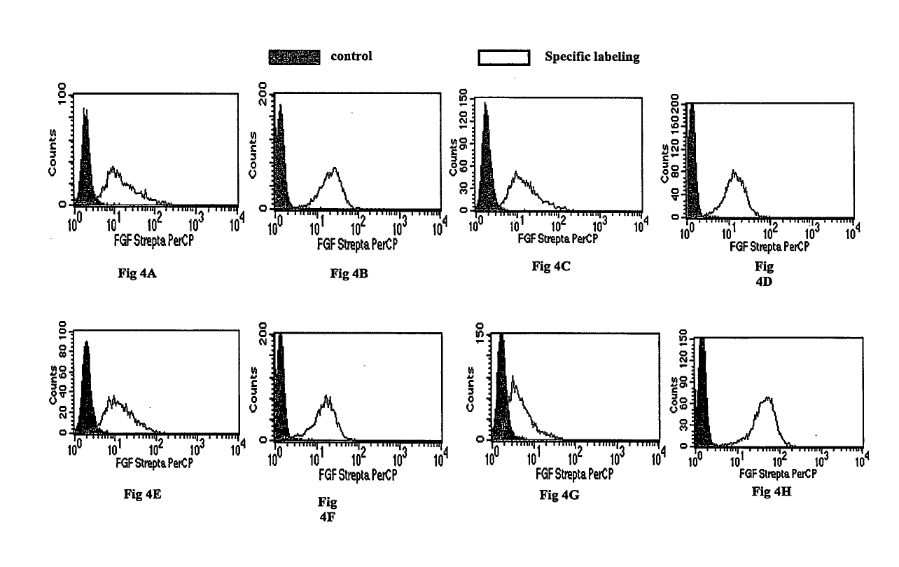

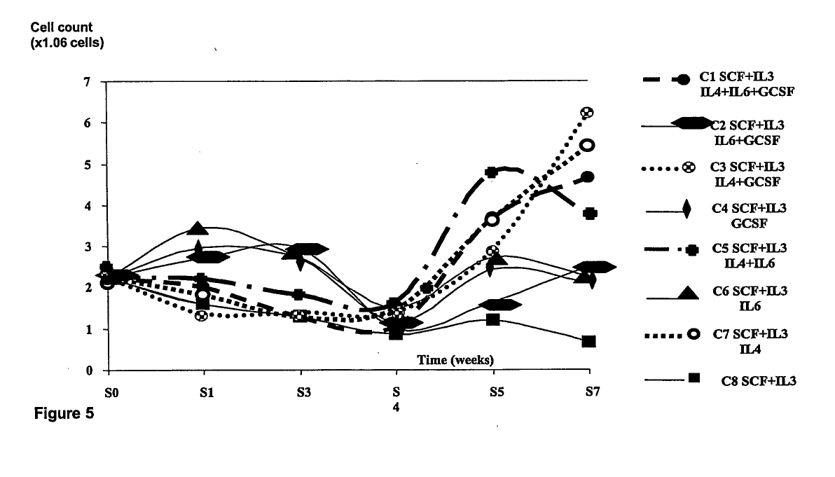

FIGS. 4A to 4H illustrate the FGF labeling of mastocytes obtained after culturing for 8 weeks, respectively under the conditions C1 to C8. The dark and light peaks correspond, respectively, to the controls and to the cells obtained under the conditions C1 to C8.

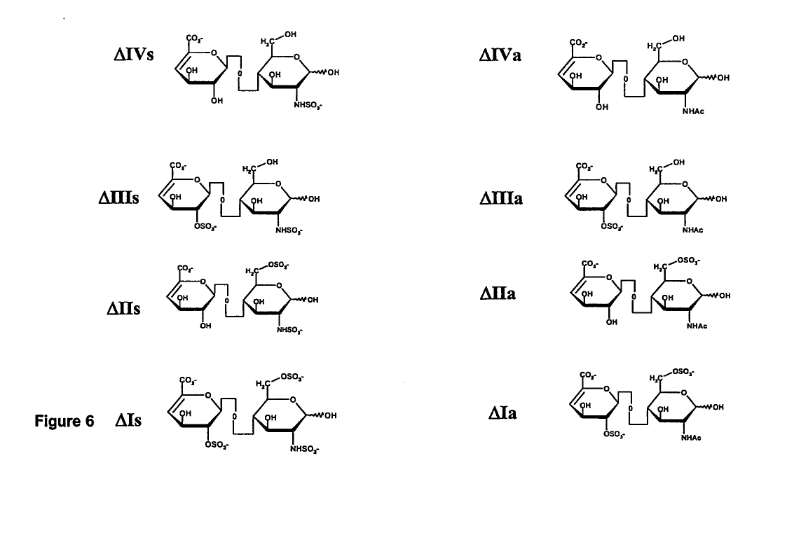

FIG. 5 illustrates the growth of the cultures under the various conditions C1 to C8 during the first 7 weeks of culturing.

FIG. 6 represents the chemical structures of the Is, IIs, IIIs and IVs disaccharides corresponding to the N-sulfated disaccharides of heparin, and also the homologous acetylated disaccharides Ia, IIa, IIIa and Iva.

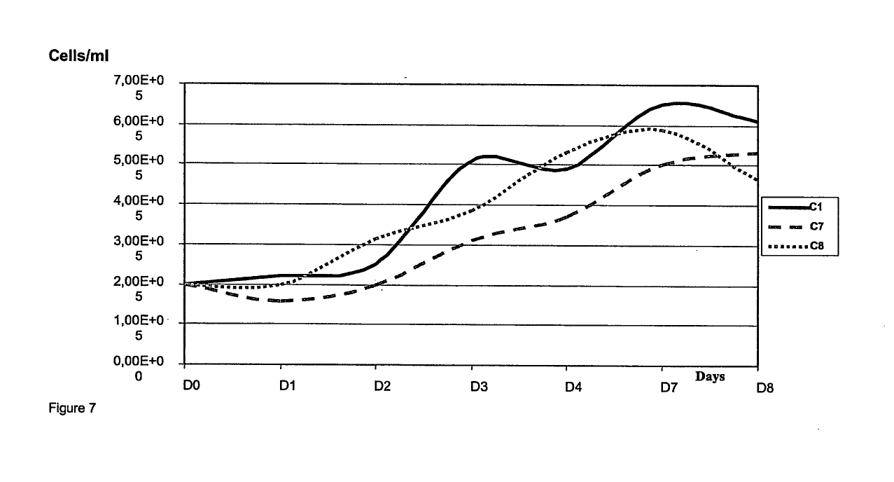

FIG. 7 illustrates the growth, in a reactor, of mastocytes obtained under conditions C1, C7 and C8.

The present invention is illustrated by the following examples of implementation. These examples are given purely by way of illustration and should not be considered as limiting.

### EXAMPLES

### Example 1

### Isolation of Mastocyte Populations from Bone Marrow of Young Pigs and Production of Lines

The animals used for taking samples are derived from protected, pig specific pathogen free (SPF) breeder colonies (MERIAL SA Lyon France). The sternums of four- and six-week-old piglets, respectively PI and PIII, are removed aseptically and then transported in a sterile container to the laboratory to be decontaminated and rinsed with, successively, a solution of pure bleach diluted to 1/100 in PBS (phosphate buffered saline, pH 7.4) buffer and then in PBS. The sternums are then cut and the bone marrow is then drawn out using a syringe, so as to then be diluted with PBS.

The medullary suspension is sieved through a sterile compress, diluted in 40 ml of PBS and then centrifuged for 10 minutes at 400 g. The cell pellet is taken up in 5 ml of PBS and then purified on 5 ml of Ficoll (Dutscher) (1100 g×10 min). The ring containing the medullary cells is recovered and then rinsed twice in PBS (14 ml, 400 g×10 min), and then taken up in 2 ml of PBS in order to be counted; approximately 1×10⁸ total cells per sternum.

After counting, the cells are centrifuged then taken up, at a concentration of 1-3×10⁶ cells/ml in 6-well culture plates and 4 ml per well, in the medium containing the following components: MEM® (Invitrogen), 15% fetal calf serum (PAA Laboratories), 100 IU/ml penicillin (Sigma), 100 μg/ml streptomycin (Sigma), 2 ng/ml porcine r-IL-3 (Biotransplant) and 80 ng/ml porcine r-SCF (Biotransplant). As soon as they are placed in culture, the cells are cultured in the culture medium described above supplemented with cytokines (1 ng/ml recombinant porcine IL-4, R&D systems; 100 ng/ml recombinant porcine IL-6, R&D systems; 10 ng/ml recombinant human G-CSF) as indicated in table 1 below.

TABLE 1
Cell culture conditions

| cytokines | C1 | C2 | C3 | C4 | C5 | C6 | C7 | C8 |
|---|---|---|---|---|---|---|---|---|
| IL-4 | + | − | + | − | + | − | + | − |
| IL-6 | + | + | − | − | + | + | − | − |
| G-CSF | + | + | + | + | − | − | − | − |

(+ medium with; − medium without cytokine)

The culture plates are incubated at 38° C.+/−0.5° and under a 5% CO₂ atmosphere.

Twice week, and for eight weeks, the medium of each well is renewed with fresh medium. The mastocyte phenotype of the isolated cells is characterized from week 2 and then at regular intervals (week 3, week 5 and week 7).

Porcine mastocyte lines, obtained under some of the conditions indicated above, were deposited with the Collection de Cultures de Microorganisms [Collection of Cultures and Microorganisms] of the Institut Pasteur (CNCM) on Apr. 9, 2003.

These lines, deposited under the numbers I-3010, I-3011, I-3012, I-3013, I-3014, were respectively obtained under the conditions C1, C2, C3, C4 and C5 described in table 1.

Confirmation of the mastocyte phenotype of the cells under each culture condition is demonstrated by detecting, by fluorocytometry, the presence of specific markers such as IgE receptor and tryptase. Detection of labeling with FGF is also carried out to reveal the site for binding of the FGF to the heparin of the mastocytes.

Labeling of Tryptase

A 1 ml sample of cell suspension from each condition is taken. Each sample is rinsed once by centrifugation in PBS buffer, and the cells are then resuspended in 400 μl of PBS buffer containing 0.5% of BSA (bovine serum albumin) and 0.01% of sodium azide.

These cells are then permeabilized at 4° C. in 200 μl per sample of a cytofix/cytoperm solution (Pharmingen) and incubated for 25 minutes. After two rinses in permawash buffer (Pharmingen), the samples are incubated for 30 minutes at 4° C. with 1 μg of tryptase-specific murine monoclonal antibody (mouse anti-human mast cell tryptase; Chemicon).

After three rinses in permawash buffer, the labeling is revealed by incubation for 25 minutes with an FITC-labeled anti-mouse immunoglobulin conjugate (FITC-conjugated affinity pure goat antimouse IgG; Jackson Immunoresearch) Duplicate samples were prepared according to the same procedure; except for the incubation with the anti-tryptase antibody, in order to be able to subtract, during analysis, the fluorescence due to the nonspecific binding of the FITC-labeled conjugate.

At the end of the final incubation, the cells are rinsed twice in permawash buffer, and then resuspended in cold PBS buffer supplemented with 1% of formaldehyde (Sigma).

The analysis by cytofluorimetry is carried out on a FACS (Faxcalibur Becton Dickinson).

Labeling of the IgE Receptor

A sample of approximately 2×10⁵ cells from each condition was taken, rinsed twice in PBS and then incubated with 2 μg per 10⁶ cells of canine IgE (Monoclonal canine IgE; Bethyl). The samples are incubated for 3 h 30 minutes at 37° C., and then rinsed twice in PBS. After having been resuspended in PBS, the samples are then incubated for 30 minutes at 4° C. with 5 μg per 10⁶ cells of goat anti-canine IgE antibody (Goat anti Dog IgE affinity purified; Bethyl).

After incubation, the samples are again rinsed twice in PBS, and then incubated for 30 minutes with an FITC-labeled anti-goat Ig conjugate (Donkey anti Goat/Sheep FITC; Serotec). After two rinses in PBS buffer, the samples are resuspended and fixed in buffer supplemented with 1% of formaldehyde. As previously, sample duplicates are also produced, omitting the incubation with IgE in order to subtract, during the analysis, the fluorescence due to the nonspecific binding of the FITC-labeled conjugate. A sample of nonmastocytic porcine cells (IRP) is also analyzed under the same conditions in order to confirm the specificity of the labeling.

Labeling of the FGF Binding Site

The cell culture samples to be analyzed are distributed in a 96-well, conical-bottomed plate, in a proportion of 0.2×10⁶ per well, and then centrifuged at 1400 rpm for 4 min. The cell pellet is rinsed in 100 μl of PBS buffer containing 5 g/l of bovine albumin and then centrifuged at 1400 rpm for 4 min. Two successive rinses are performed under the same conditions.

The cell pellets are diluted in a Cytofix/Cytoperm fixing/permeabilizing buffer (Pharmingen), rinsed in 100 μl of Perm/Wash buffer (Pharmingen) and then centrifuged at 1400 rpm for 4 min at 4° C. Three successive rinses are performed under the same conditions.

The cell pellets are diluted in 100 μl of Perm/Wash buffer containing 172 ng/ml of basic FGF (R&D systems) and incubated for 30 minutes in ice. The cells are rinsed in 100 μl of Perm/Wash™ buffer and then centrifuged at 1400 rpm for 4 min at 4° C. Three successive rinses are performed under the same conditions.

The cell pellets are diluted in 100 μl of Perm/Wash buffer containing 1 μg of biotin-coupled anti-basic FGF mouse monoclonal antibodies (R&D systems) and incubated for 30 minutes in ice. The cells are rinsed in 100 μl Perm/Wash buffer and then centrifuged at 1400 rpm for 4 min at 4° C. Three successive rinses are performed under the same conditions.

The cell pellets are diluted in 100 μl of Perm/Wash buffer containing a solution of streptavidin peridinin chlorophyll-a protein and incubated for 20 minutes in ice in the dark. The cells are rinsed in 100 μl of Perm/Wash™ buffer and then centrifuged at 1400 rpm for 4 min at 4° C. Three successive rinses are performed under the same conditions. The pellet is diluted in 150 μl of PBS buffer containing 5 g/l of bovine albumin, 0.01% of sodium azide and 1% formaldehyde. The presence of the intracytoplastic labeling is detected by cytofluorimetry.

Sample duplicates are prepared according to the same procedure, except for the incubation with the anti-FGF antibody, in order to be able to subtract, during the analysis, the fluorescence due to the nonspecific binding of the FITC-labeled conjugate. A sample of nonmastocytic porcine cells (IPR) is also analyzed under the same conditions in order to confirm the specificity of the labeling.

FIGS. 1, 2, 3 and 4 show the results of the phenotypic characterization of the mastocytes obtained, respectively, after culturing for 3, 7 and 8 weeks.

Positive and specific labeling of the cells for the mastocyte markers, IgE receptor and tryptase, and also detection of intracellular binding of the FGF, are observed. The detection carried out on the cells from the third week of culturing is positive for the presence of tryptase. The cultures under conditions 1 to 5 are homogeneous and contain 100% of mastocytes, as revealed by labeling of the IgE receptor from week 5.

In week 7, the cultures under conditions C1 to C5 and C7 are 100% homogeneous, the homogeneity of the cultures under conditions C6 and C8 is greater than 50%.

Electron Microscopy Analysis

The characterization of the isolated cells was also completed by electron microscopy observation. The cells exhibit a morphology characteristic of mastocytes, with many granulations, with a large off-center nucleus, and with an uneven outline.

Cell Proliferation During the Isolation

At regular intervals, culture samples for each condition (C1 to C8) are taken and counted under the microscope after dilution in PBS buffer supplemented with 0.4% of trypan blue.

A decrease in the cell concentration during the first four weeks of culturing (W1 to W4), corresponding to the death and the lysis of the medullary cells not stimulated by the SCF and to the passing from a heterogeneous culture to an essentially mastocytic culture, is observed. From the fifth week (W5), proliferation of the cultures is observed, correlated with more intense labeling of the mastocyte-specific markers. The proliferation is substantially greater for the culture conditions comprising IL-4 (FIG. 5).

Characterization of the Heparin Content of the Cultures by HPLC

After culturing and amplifying for 15 weeks, samples were taken in order to analyze the proteoglycan composition of the mastocytes according to the protocol described by Linhardt and al (Biomandhodes, 9, 183-197, 1997). The samples are treated in the following way:

Proteolysis: The cell samples, 2×10⁶ cells, are centrifuged and rinsed twice in PBS buffer. Each pellet is taken up in 100 μl of distilled water supplemented with 10 μl of alcalase (Novozymes) and then heated for 5 hours at 60° C. with agitation. The samples are then diluted with 200 μl of 10 mM Tris buffer, pH 7.0 (Prolabo) containing 0.5 M NaCl (Prolabo), before being centrifuged for 10 minutes at 10,000 rpm. The proteolysis step makes it possible to release the intracellular content and to dissociate the protein-polysaccharide bonds.

Extraction: The supernatant of each sample is purified by ion exchange on SAX quaternary ammonium resin in a 96-well plate format, 100 mg/2 ml (Thermohypersil). After binding and washing in Tris buffer, pH 7, containing 0.5 M NaCl, the glycoaminoglycans (GAGs) are eluted with 500 μl of Tris buffer, pH 7.0, containing 3 M NaCl.

Desalification/concentration: The samples are then desalified on a gel permeation column (NAP-5, Pharmacia). After elution in a volume of 1 ml, the samples are concentrated by lyophilization and then taken up in 130 μl of distilled water.

Depolymerization: For the HPLC analysis, the GAGs are depolymerized with a mixture of heparinases I, II and III (Grampian enzymes). Each heparinase solution is adjusted to 0.5 IU/ml in phosphate buffer. The solution of heparinases I, II, III is prepared by mixing ⅓ volume for volume of each heparinase solution. For 100 μl of sample to be analyzed, 15 μl of the heparinase mixture and 10 μl of acetate buffer containing 0.73 ml of 100% acetic acid (Prolabo), 12.5 mg of bovine albumin (Sigma) and 39.5 mg of calcium acetate (Prolabo) per 30 ml of distilled water were added.

HPLC analysis: The samples are then analyzed by HPLC on a Waters spherisorb SAX 5 μm, 250×3 mm, Thermohypersil column. 50 μl of sample are injected per analysis; the mobile phase buffer is composed of 2.5 mM sodium dihydrogen phosphate (Na₂HPO₄, Prolabo), the pH of which is adjusted to 2.9 with ortho-phosphoric acid (H₃PO₄, Prolabo). The elution of the disaccharides constituting the GAGs extracted from the cell samples is carried out in a gradient of 0 to 100%, in 50 minutes, of 2.5 mM Na₂HPO₄ buffer containing 1 M of perchlorate (NaClO₄, Prolabo). The disaccharides are detected via their retention time and relative to a standard heparin sample (Aventis), by UV at 234 nM.

Analysis of the cell cultures after 15 weeks of culturing reveals the presence of large amounts of heparin-type compounds in the cells, confirming the specifically mastocytic nature of the isolated cultures.

The major disaccharides constituting heparin, as described by Linhardt et al. (Biomandhodes, 9,183-197, 1997), are in fact found.

These disaccharides are mainly represented by Is, IIs, IIIs and IVs (chemical structures represented in FIG. 6) corresponding to the N-sulfated disaccharides of heparin. The homologous acetylated disaccharides IIa, IIIa and Iva (FIG. 6) are also found.

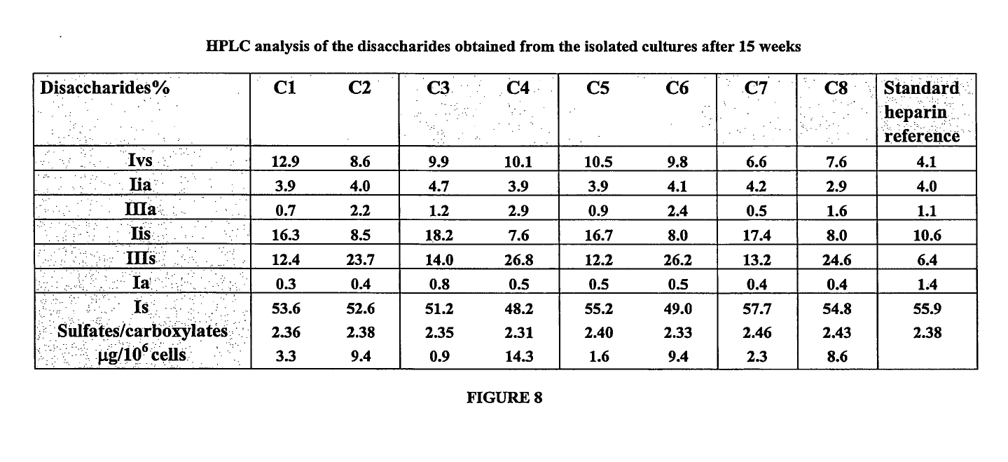

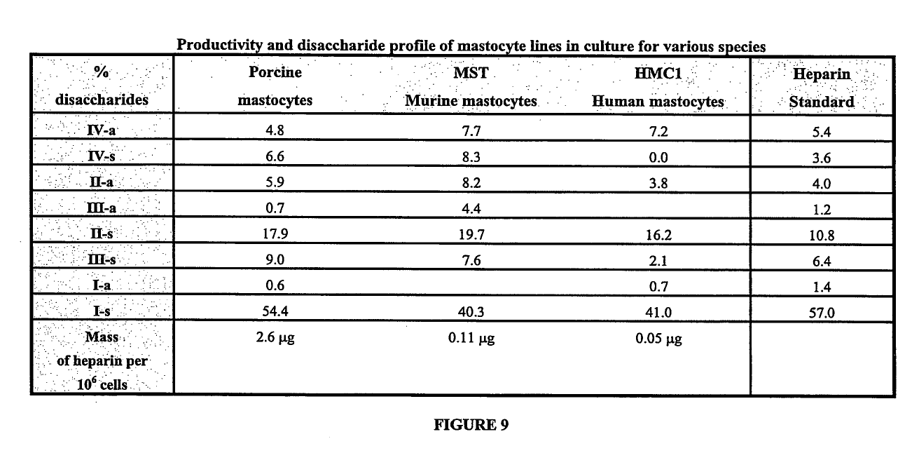

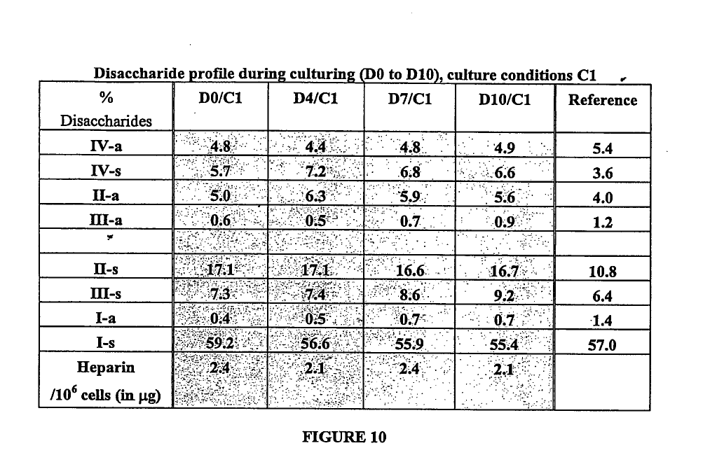

The table presented in FIG. 8 gives the compositions obtained for each culture.

A reproducible modulation of the disaccharide structure is observed as a function of the presence or absence of IL-4, this modulation is mainly observed on the percentage of IIs and IIIs disaccharides.

### Example 2

### Culturing of the Lines and Analysis of the Production of Heparin-Type Molecules

The disaccharide profile of the isolated mastocytes was analyzed for three culture medium conditions (C1, C7 and C8). The cells were amplified in suspension and cultured in a 100 ml spinner.

Cell Culture

The initial cell density is 2×10⁵ cell per ml; the cells are incubated under a 5% C02 atmosphere at 37° C. and counting is performed under a microscope at regular intervals.

Samples of the cultures thus produced are taken at the time of the counting, for HPLC analysis of the heparin-type polysaccharide content and measurement of the anti-IIa and anti-Xa biological activity.

Under these conditions, the maximum cell density is between 4 and 6×10⁵ cells/ml, with an exponential doubling time of between 24 and 48 hours.

FIG. 7 illustrates the growth of the mastocytes at the 14^{th} passage.

Analysis of Polysaccharides

The HPLC analysis of the samples for three harvesting days (D4, D7 and D10) shows, for all the cultures, a heparin-type profile for the polysaccharides, with IL-4 having an effect on the relative percentage of the IIs and IIIs disaccharides. The productivity of the cultures is significant, between 2 and 12 μg for 10⁶ cells.

By comparison, the cultures of mastocyte lines of the murine species, such as the MST cells described by Montgomery et al (Proc. Natl. Acad. Sci., 89, 11327-11331, 1992), or the human mastocyte line HMC1 (Bufterfield et al Leuk Res, 12(4), 345-355, 1988) exhibit a heparin-type compound productivity of 20 to 200 times less than the porcine lines which are the subject of the present invention (tables 3 and 4).

Biological Activity of the Polysaccharides

Inactivation of factors Xa and IIa is characteristic of heparin and makes it possible to differentiate it from heparan sulfate and from dermatan. The method used is that described in the monograph of the European Pharmacopoeia, 3^{rd} edition (1997).

The reaction takes place in three steps:

1: ATIII+heparin [ATIII-heparin]

2: [ATIII-heparin]+factor in excess+residual factor [ATIII-heparin-factor]

3: Residual factor+chromophore substrate Colored para-nitroanilin

The amount of para-nitroaniline released is inversely proportional to the amount of heparin.

The anti-Xa or anti-IIa amount is measured relative to a calibration line established with the SPIM standard (Standard International Heparin). The sensitivity of the method is 0.006 IU/ml.

The biological activity is expressed in IU/mg, taking into account the quantification of the disaccharides obtained by HPLC.

The analysis carried out on the 10^{th} day of harvesting after the end of the growth phase reveals an anti-Xa and anti-IIa biological activity of between 10 and 25 IU/mg. It is noted that, for this stage of the culture, the ratio between the two activities is close to 1, which is the ratio characteristic of the heparin derived from extraction from pig intestinal mucosa. The results obtained by measuring inactivation of the factors Xa and IIa are summarized in table 5.

TABLE 5
Measurement of the inactivation of factors Xa and IIa

| | Condition | | |
|---|---|---|---|
| | C1 | C7 | C8 |
| Anti-IIa activity | 12 | 12 | 26 |
| (IU/mg) | | | |
| Anti-Xa activity | 12 | 12 | 26 |
| (IU/mg) | | | |

### Example 3

### Genetic Modification of the Isolated Cells

The mastocytes can be genetically modified by introducing an exogenous nucleic acid using, for example, transfection, electroporation, nucleoporation or infection techniques, which will result in transient or stable expression of the nucleic acid introduced. In the case of stable expression, the DNA may be integrated into the cell genome or may be maintained as an episome.

1. Transfection by Nucleoporation and Electroporation

Stably transfected cells can be obtained using the nucleoporation method described below, applying, 24 to 72 hours after nucleoporation, a selection pressure (hygromycin, geneticin, blasticidin, puromycin or zeocin). The resistance to the selection agent is conferred by the integration of the plasma carrying the gene of interest and the resistance gene.

Nucleoporation

This method is preferentially used since it makes it possible to target the DNA directly into the nucleus.

1 to 2×10⁶ mastocytes, in the exponential phase, preferentially after 3 or 4 days of culturing, are centrifuged at 1000 rpm for 5 minutes and taken up in 100 μl of nucleofection solution (Amaxa, Kit 8351). 2 to 4 μg of pcDNA3.1-eGFP, a plasmid encoding GFP, are then added to the cell suspension. The cells are then transferred into the electroporation cuvette and subjected to an electric shock using a specific program (such as U14, T20 and T22 AMAXA).

The cells are then transferred into 2 ml of complete medium preheated to 37° C., and are then incubated at 37° C., 5% CO₂.

24 to 48 hours after the transfection, the cells are harvested in order to be fixed with 1% paraformaldehyde (Prolabo). For this, the entire culture is centrifuged for 5 min at 1000 rpm. After removal of the supernatant, the cells are washed in 4 ml of 1×PBS and then centrifuged again. The cell pellet is then taken up in 1 ml of 1% paraformaldehyde. The cells thus fixed are then analyzed in a cytometer (Cytomics FC 500, Beckman Coulter)

The nucleoporation conditions described above make it possible to transfect the pig mastocytes with a transfection efficiency of between 30 and 50%, while at the same time obtaining good cell viability, greater than 50%.

Electroporation

1 to 5×10⁶ cells, in the exponential phase, are brought into contact with 1 to 30 μg of DNA. The cells, transferred into a 4 mm electroporation cuvette, are incubated for 5 min in ice before being electroporated at a voltage of between 150 V and 400 V with a capacitor of 500 or 960 μF (Gene Pulser II, Biorad). After electroporation, the cells are again incubated for 5 min in ice and are then finally transferred in 5 ml of complete culture medium and incubated at 37° C., 5% CO₂.

The process for selecting cells which have integrated the transgene stably uses the same technique as described above, using the resistance, conferred by the integration of the plasmid, to a selection agent.

2. Transfection with Viral Vectors, Use of Pantropic Retroviral Vectors

As an alternative to the methods of transfection by electroporation and nucleoporation, use may be made of replication-deleted recombinant retroviral vectors. Use may, for example, be made of vectors pseudotyped with the vesicular stomatitis virus envelope glycoprotein (VSV-G) which allows production of pantropic retroviral vectors capable of infecting porcine cells.

In this method of transfection, the retroviral vector carrying the gene of interest to be expressed in or integrated into the porcine mastocyte is produced, initially, using the packaging cell such as GP-293 (Clontech protocol ref PT 3132-1), which contains the genetic elements for producing the vector (gag and pol) with the exception of the gene for the production of the pseudotyped envelope protein (env-VSV-G).

At the time of production of the retroviral vector, the packaging cells are cotransfected with the plasmid encoding the VSV-G envelope gene and a retroviral plasmid encoding the gene of interest under the control of a promoter with or without a selection gene.

In practice, the GP-293 cells are placed in culture for 48 to 72 hours before transfection in order to be in the exponential growth phase. On the day of transfection, the culture medium is replaced with fresh medium (15-20 ml per 10⁶ cells), and then 1 to 2 ml of solution containing the mixture of VSV-G plasmid (5 to 20 μg per 10⁶ cells) and the plasmid carrying the gene of interest (10 to 30 μg per 10⁶ cells), in a calcium phosphate buffer, pH7, are added dropwise to the culture medium (1 to 2 ml (Promega)).

The cells are then incubated again, for 16 to 24 hours at 37° C. or preferably at a temperature of between 32 and 35° C. The culture medium is again replaced with fresh medium. The cells are incubated for a further 48 hours at 32-35° C. At the end of the incubation period, the culture supernatant containing the newly formed retroviral vectors is harvested. Part of the supernatant from infection of the packaging cells is aliquotted and frozen at −80° C., the other part is mixed with the culture medium of the mastocytes in the exponential growth phase. In practice, the mastocytes in culture are centrifuged and resuspended in a medium containing 50% of fresh medium and 50% of infection supernatant. The mastocytes are incubated for 24 hours at 32-35° C., and the medium is then again replaced with fresh medium.

48 to 72 hours after the infection of the mastocytes, samples are taken to be analyzed by cytofluorimetry in the case of the control involving infection with the GFP (Green Fluorescent Protein) fluorescent reporter or by PCR for the infections with the gene of interest. By cytofluorimetry, the measurement for the efficiency of transfection is greater than 20% of the total cells.

The transfected cell populations are then selected by adding to the culture medium the cytotoxic agent (Hygromycin, puromycin, G418, Zeocin) for which only the mastocytes transfected with the retroviral vector carrying the gene of interest and the resistance gene continue to grow. Through this method, the gene of interest is stably integrated and is stably expressed.

After selection and cloning of the populations, the selection agent can be removed from the culture medium while at the same time conserving the expression of the gene of interest. The retroviral vector produced in this way does not replicate in the host mastocyte and there is therefore no production of replicative vectors.

Alternatively, use may be made of vectors for which the expression is subjected to induction of the promoter regulating expression of the gene of interest by a compound added to the culture medium at the desired time.

### Example 4

### Isolation of the Porcine c-Kit Gene

The porcine c-kit gene was isolated by 3′-RACE using, as RNA source, total RNA isolated from a pig liver mastocyte culture according to the previously published procedure (Piu et al, CR Acad. Sci. Paris, 316, 772-779, 1993).

Reverse transcription of 2 μg of total RNA to cDNA was carried out according to the protocol of the 5′/3′ RACE kit (Roche), using, as primer, an oligodT, called OligodT anchor primer, of sequence SEQ ID NO. 8 ^{5′}gac cac gcg tat cga tgt cga ctt ttt ttt ttt ttt ttv^{3′}. The CDNA is then amplified by PCR using the protocol of the Expand High fidelity system kit (Roche).

The PCR was carried out on 1 μl of CDNA, with the sense primer C15203, which hybridizes specifically in the noncoding 5′ region of the porcine c-kit gene (nucleotides 24 to 42 relative to the published porcine c-kit sequence, GenBank ref AJ223228) of sequence SEQ ID NO. 9 ^{5′}gga att cct cga gag cag gaa cgt gga aag gag^{3′} and the antisense primer, called PCR anchor primer, of SEQ ID NO. 10 ^{5′}gac cac gcg tat cga tgt cga c^{3′}, which hybridizes specifically in the 3′ position at the level of the oligo dT primer. 10 PCR cycles and then 25 PCR cycles were applied (cycle 1: 15 sec of denaturation at 94° C., 45 sec of hybridization at 55° C. and 4 min of elongation at 68° C., cycle 2: 15 sec of denaturation at 94° C., 45 sec of hybridization at 60° C. and 4 min of elongation at 68° C.).

The PCR product obtained is purified on a 1% agarose, 1×TBE gel, using the Quiaquick gel extraction kit (Quiagen).

A second PCR, identical to the first, is carried out on 1/30^{th} of the purified PCR product, by applying 25 thermal cycles (15 sec of denaturation at 94° C., 45 sec of hybridization at 60° C. and 4 min of elongation at 68° C.). At the end of the second PCR, the PCR product is again purified in order to clone it into a vector, pGEMTeasy, according to the pGEM-T Easy vector system (Promega) protocol.

The sequence of the porcine gene is then partially determined by sequencing. The nucleotide sequence obtained is the sequence SEQ ID NO. 1. The deduced protein sequence is the sequence SEQ ID NO. 2. This sequence SEQ ID NO. 1 shows differences compared to the sequence published under the reference AJ 223228. Specifically, the C-terminal end has 9 additional amino acids and the following differences in the nucleotide sequence were observed, leading to the modification of two amino acids:

Modifications (compared to the published sequence AJ 223228):
nt 237 t→g: H→Q
nt 351 a→t
nt 523 a→c
nt 606 c→t
nt 609 g→a
nt 635 g→a: R→K
nt 639 c→t
nt 663 c→g
nt 669 c→a
nt 2016 a→g
nt 2865 c→t

### Example 5

### Isolation and Sequencing of the 3′ Coding Sequence of the Porcine 3-OST Gene

The partial sequence of the porcine gene encoding 3-OST is available in an EST library (GenBank accession number BF075483). Alignment of this sequence with the human sequence shows that it lacks approximately 650 bp of the 3′ coding region.

The missing portion of the porcine 3-OST gene was identified by combining RT-PCR and 3′-RACE using, as RNA source, pig liver RNAs isolated according to the protocol of the Trizol kit (Invitrogen).

Reverse transcription of 2 μg of total RNA to cDNA was carried out according to protocol of the First Strand Synthesis System kit (Invitrogen), using, as primer, a mixture of the oligonucleotides BS02 and BS03 of respective sequences SEQ ID NO. 11 5′-GCA GCA GCC ACG TCG GG-3′ and SEQ ID NO. 12 5′-TCA GTG YCA GTC RAA TGT TC-3′.

2 μl of these cDNAs were then amplified by PCR in the presence of a sense primer, BS05, of sequence SEQ ID NO. 13 5′-CGG NGA CCG CCT NAT CAG-3′ and of an antisense primer, BS06, of sequence SEQ ID NO. 14 5′-TCA GTG YCA GTC RAA TGT TC-3′ with the KOD hot start polymerase (Novagen). After the 30 thermal cycles (15 sec of denaturation at 98° C., 30 sec of hybridization at 60° C. and 30 sec of elongation at 68° C.), the amplified fragment of 277 bp was cloned into the vector pCR-Blunt II TOPO (Invitrogen, Zero Blunt TOPO PCR Cloning kit) and then sequenced.

The sequence of this fragment was used to generate 2 primers BS21 and BS22, in order to isolate, by 3′-RACE, the entire 3′ region.

Within the framework of the 3′-RACE, 1 μl of porcine liver RNA was reverse transcribed to cDNA according to the protocol of the First Strand Synthesis System kit from Invitrogen, using, as primer, the oligodT CDSIII of SEQ ID NO. 15 (5′-ATT CTA GAG GCC GAG GCG GCC GAC ATG T₃₀ VN-3′).

The 3′-region of the gene encoding 3-OST was then amplified by 2 successive PCRs. The first PCR was performed on 2 μl of cDNA, obtained previously, with the sense primer BS21 of SEQ ID NO. 16 5′-GCA CCC CCA GAT CGA CCC C-3′ and an antisense primer CDSIII. 30 thermal cycles were applied (10 sec of denaturation at 94° C., 30 sec of hybridization at 60° C. and 120 sec of elongation at 68° C.). The second PCR was then carried out under the same conditions as the first PCR, with 1 μl of product derived from the first PCR, using the sense primer BS22 of sequence SEQ ID NO. 17 5′-CAA ACT CCT CAA TAA ACT GCA CG-3′ and the antisense primer CDSIII.

Sequencing of the PCR product thus obtained at the end of the 3′-RACE made it possible to identify the 3′ sequence of the porcine 3-OST and also approximately 250 bp of the noncoding region.

Isolation of the Complete Coding Phase of the Porcine 3-OST Gene

In order to clone the complete coding phase of the porcine 3-OST, a further RT-PCR experiment was carried out using the information obtained in the first step. The source of RNA is the same as in the preceding step.

2 μg of RNA were reverse transcribed to cDNA according to the protocol of the First Strand Synthesis System kit from Invitrogen, using, as primer, an oligonucleotide dT₂₄.

The gene encoding 3-OST was then amplified by PCR in two steps. The first PCR made it possible to amplify the gene, including a portion of the 3′ noncoding sequence of the gene, the second PCR then made it possible to amplify the coding sequence using primers compatible with the Gateway system (Invitrogen).

The first PCR was carried out on 2 μl of cDNA with a sense primer BS10 of sequence 5′-AGG CCC GTG ACA CCC ATG AGT-3′, which hybridizes specifically in the 5′ noncoding region of the porcine 3-OST gene, and an antisense primer BS30 of sequence 5′-CAC CTA GTG TAC ACC ACA ATT TAC-3′, which hybridizes specifically in the 3′ position at the level of the UTR. 35 thermal cycles were applied (10 sec of denaturation at 98° C., 30 sec of hybridization at 64° C. and 150 sec of elongation at 68° C.).

A second PCR was carried out on 1 μl of PCR product in order to specifically amplify the coding phase. For this, we used the sense primer BS31 of sequence SEQ ID NO. 18 ^{5′}GGG GAC AAG TTT GTA CAA AAA AGC AGG CTC AGC ATG GCC GCG CTG CTC^{3′} and the antisense primer BS32 of sequence SEQ ID NO. 19 ^{5′}GGG ACC ACT TTG TAC AAG AAA GCT GGG TTT AGT GCC AGT CAA ATG TTC TGC C^{3′}. The PCR program used is identical to that used for the first PCR.

The PCR product of 1 kb was then cloned, according to the procedure of the Gateway cloning technology kit, invitrogen, into the episomal vector pE-IRES-neo2. The sequence of the porcine gene was verified by sequencing. The nucleotide sequence obtained is the sequence SEQ ID NO. 4. The deduced protein sequence is the sequence SEQ ID NO. 5.

### Example 6

### Identification of the Complete Coding Sequence of the Porcine 6-OST Gene

The partial sequence of the porcine gene (nucleotide 682 to 910 of the human sequence) encoding 6-OST is available in an EST library (GenBank accession number BE235545).

The complete coding sequence of the 6-OST gene was identified by combining two RT-PCR experiments with 5′ and 3′-RACE experiments using, as RNA source, pig liver RNAs isolated according to the protocol of the Trizol kit (Invitrogen).

Reverse transcription of 80 ng of total RNA to cDNA was carried out according to the protocol of the First Strand Synthesis System kit (Invitrogen), using, as primer, an oligonucleotide dT24.

2 μl of these cDNAs were then amplified by PCR in the presence of a sense primer 386-03 of sequence SEQ ID NO. 20 5′-AGA TGA CTG GTC GGG CTG C-3′ and of an antisense primer 386-01 of sequence SEQ ID NO. 21 5′-CAA TGA TRT GGC TCA TGT AGT CC-3′ with the KOD hot start polymerase (Novagen). After the 35 thermal cycles (15 sec of denaturation at 95° C., 30 sec of hybridization at 60° C. and 2 min of elongation at 68° C.), the amplified fragment of 537 bp was cloned into the vector pCR-Blunt II TOPO (Invitrogen, Zero Blunt TOPO PCR Cloning kit) and then sequenced.

The sequence of this fragment was used to generate three primers, 386-05, 386-19 and 386-20, used for the next PCR and the 3′-RACE.

2 μl of the cDNAs previously obtained were amplified by PCR in the presence of a sense primer, 386-07, of sequence SEQ ID NO. 22 5′-ATG GTT GAG CGC GCC AGC AAG TTC G-3′ and of the antisense primer 386-05 of sequence SEQ ID NO. 23 5′-GGT TAT TGG CCA GGT TGT AGG GGC-3′ with the KOD hot start polymerase (Novagen). After the 30 thermal cycles (15 sec of denaturation at 95° C., 30 sec of hybridization at 60° C. and 1 min of elongation at 68° C.), the amplified fragment of 718 bp was cloned into the vector pCR-Blunt II TOPO (Invitrogen, Zero Blunt TOPO PCR Cloning kit) and then sequenced.

The sequence of this fragment was used to generate two primers, 386-24, 386-26, used for the 5′-RACE.

Within the framework of the 3′-RACE, 1 μl, of porcine liver RNA was reverse transcribed to cDNA according to the protocol of the First Strand Synthesis System kit from Invitrogen, using, as primer, the oligodT CDS-C of sequence SEQ ID NO. 24 5′-ATT CTA GAG GCC GAG GCG GCC GAC ATG T₃₀ VC-3′.

The 3′ region of the gene encoding 6-OST was then amplified by 2 successive PCRs. The first PCR was carried out on 2 μl of cDNA, previously obtained, with the sense primer 386-19 of sequence SEQ ID NO. 25 5′-GGA CCT CTT CCA GCA GCG-3′ and the antisense primer CDS-C with the Advantage 2 polymerase mix (Clontech). 24 thermal cycles were applied (7 sec of denaturation at 98° C., 10 sec of hybridization at 62° C. and 2 min of elongation at 68° C.). A second PCR was then carried out on 2 μl of product derived from the first PCR, using the sense primer 386-20 of sequence SEQ ID NO. 26 5′-GCT ATC AGT ACA AGC GGC AGC-3′ and the antisense primer CDS-C. After the 30 thermal cycles (7 sec of denaturation at 95° C., 10 sec of hybridization at 62° C. and 2 min of elongation at 68° C.), the amplified fragment of 300 bp was cloned into the vector pCR-Blunt II TOPO (Invitrogen, Zero Blunt TOPO PCR Cloning kit) and then sequenced. This experiment made it possible to identify the 3′ coding region for 6-OST and approximately 32 bp of the noncoding region.

Within the framework of the 5′-RACE, 2 μl of porcine liver RNA were reverse transcribed to cDNA according to the protocol of the First Strand Synthesis System kit from Invitrogen, using, as primer, the oligonucleotide 386-28 of sequence SEQ ID NO. 27 5′-CCA GGC TCA GCC CCG G-3′.

The phosphorylated oligonucleotide okib57 of sequence SEQ ID NO. 28 5′-p GTA GGA ATT CGG GTT GTA GGG AGG TCG ACA TTG CC-3′ was grafted 5′ of the cDNA by ligation (RNA ligase, Roche).

The 5′ region of the gene encoding 6-OST was then amplified by 2 successive PCRs. The first PCR was carried out on 2 μl of grafted cDNA, with the sense primer okib58 of sequence SEQ ID NO. 29 5′-GGC AAT GTC GAC CTC CCT ACA AC-3′, which hybridizes to the primer okib57, and the antisense primer 386-24 of sequence SEQ ID NO. 30 5′-TCA GCC CCG GGC CCG CG-3′ according to the protocol of the Advantage 2 polymerase mix kit. 24 thermal cycles were applied (10 sec of denaturation at 98° C., 10 sec of hybridization at 64° C. and 2 min of elongation at 72° C.). A second PCR was then carried out on 0.5 μl of product derived from the first PCR, using the sense primer okib59 of sequence SEQ ID NO. 31 5′-CTC CCT ACA ACC CGA ATT CCT AC-3′ and the antisense primer 386-26 of sequence SEQ ID NO. 32 5′-GCC CGC GTA CTG GTA GAG G-3′. After the 40 thermal cycles (10 sec of denaturation at 98° C., 10 sec of hybridization at 66° C. and 2 min of elongation at 72° C.), the amplified fragment of 170 bp was sequenced. This experiment made it possible to identify the 5′ coding region for 6-OST and approximately 14 bp of the noncoding region.

Isolation of the Complete Coding Phase of the Porcine 6-OST Gene

In order to clone the complete coding phase of the porcine 6-OST, a further RT-PCR experiment was carried out using the information obtained in the first step.

The source of RNA is the same as in the preceding step.

2 μg of RNA were reverse transcribed to cDNA according to the protocol of the First Strand Synthesis System kit, using, as primer, the oligonucleotide dT CDSIII. The gene encoding 6-OST was then amplified by PCR using primers compatible with the Gateway system (Invitrogen). The PCR was carried out on 2 μl of cDNA with a sense primer, 386-33, sequence SEQ ID NO. 33 5′-GGG GAC AAG TTT GTA CAA AAA AGC AGG CTT AGG ACA ATG GTG ACA CAT GCG GCG GC-3′ and an antisense primer 386-34 of sequence SEQ ID NO. 34 5′-GGG GAC CAC TTT GTA CAA GAA AGC TGG GTC CTA CCA CTT CTC GAT GAT GTG GCT C-3′. 35 thermal cycles were applied (5 sec of denaturation at 98° C., 20 sec of hybridization at 66° C. and 1 min 30 sec of elongation at 72° C.).

The PCR product of 1 kb was then cloned, according to the procedure of the Gateway cloning technology kit, Invitrogen, into the episomal vector pE-IRES-neo2. The sequence of the porcine gene was verified by sequencing.

The nucleotide sequence obtained is the sequence SEQ ID NO. 6. The deduced protein sequence is the sequence SEQ ID NO. 7.

### Example 7

### Transformation of the Lines According to the Invention with the Porcine c-Kit Gene

In order to obtain pig mastocytes whose growth would be SCF independent in the long term, the mastocytes can be transformed with the mutated c-kit gene.

For this, the preferentially porcine c-kit gene carrying a point mutation responsible for modification of the valine 556 to glycine (gene referred to as c-kit^{G556}) can be used, this mutation is similar to the c-kit^{G559} mutant in mice and the c-kit^{G560} mutant in humans. Alternatively, use may be made of the c-kit gene in which the amino acids TQLPYDH 570 to 576 are deleted; in mice, this deletion is similar to amino acids 573 to 579, in humans 574-580. Similarly, due to the inter-species conservation of the c-kit gene, the murine, human or bovine genes, or any other gene having at least 80% homology with the porcine gene, can be used. In this case, it is then also possible to use a point mutation responsible for modification of the aspartic acid to valine 814 or 816, respectively, in mice and in humans.

The mastocytes are transfected via one of the methods described in example 4, preferentially nucleoporation, with an integrative vector in which the coding phase of the mutated c-kit gene is cloned under the control of a strong viral (CMV, RSV) or cellular (EF1α) promoter. In addition to the mutated c-kit gene, this vector may also carry a gene encoding resistance to an antibiotic (geneticin, hygromycin, puromycin, etc.).

48 hours after transfection, the cells are counted, centrifuged and seeded at 2×10⁵ C/ml in the complete culture medium supplemented with the selection antibiotic. The cells are cultured in the presence of selection for 2 to 3 weeks, which makes it possible to eliminate the cells which are not stably transfected. After this period of selection, the cells are amplified.

The cells are then analyzed genetically by PCR and RT-PCR in order to verify the integration of the mutated c-kit gene and its expression. The independent nature of the cells with respect to SCF is demonstrated by comparing the growth of the cells transfected to the mutated c-kit gene, in an SCF-free medium, with the growth of the cells transfected with the empty vector, in a medium with and without SCF.

A variant to this protocol consists in using a vector carrying only the mutated c-kit gene. In this case, the cells are selected 48 hours after transfection without using a selection agent, but by seeding the cells at 2×10⁵ C/ml in a medium lacking SCF. The nontransfected cells are not capable of growing in a medium lacking SCF, unlike the transfected cells.

### Example 8

### Transfection of the Lines According to the Invention with the Porcine 3-OST Gene

In order to increase the biological activity of the heparin-type compounds derived from the mastocyte cultures, it is possible to stably overexpress the gene encoding 3-OST-1 (3 O-sulfatase-1).

For this, the porcine gene may be used. Alternatively, it is possible to use genes from other species, encoding expression of 3-O-sulfatase activity and exhibiting at least 80% homology with the porcine gene, in particular murine-3-OST-1.

The mastocytes are transfected by the nucleoporation method described in example 4, with an integrative plasmid in which the coding phase of the 3-OST gene has been cloned under the control of a strong viral (CMV, RSV) or cellular (EF1α) promoter. In addition to the 3-OST gene, this plasmid carries a gene encoding resistance to an antibiotic (geneticin, hygromycin, puromycin, etc.).

48 hours after transfection, the cells are counted, centrifuged and seeded at 2×10⁵ C/ml in the complete culture medium supplemented with the selection antibiotic. The cells are cultured in the presence of selection for 2 to 3 weeks, which makes it possible to eliminate the cells which are not stably transfected. After this period of selection, the cells are amplified.

The cells are analyzed genetically by PCR and RT-PCR in order to verify the integration of the mutated c-kit gene and its expression.

The functionality of the 3-OST is demonstrated by HPLC analyses of the heparin produced by the mastocytes, compared with that produced by the nontransfected mastocytes. Analyses of the biological activity of the product make it possible to confirm the increase in biological activity with respect to factor Xa and factor IIa.

1. A method for obtaining mastocytes, the method comprising the step of culturing porcine bone-marrow stem cells in a culture medium comprising at least about 0.2 ng/ml of IL-3, at least about 8 ng/ml of SCF, at least about 0.1 ng/ml of IL-4, and at least one mediator selected from the group consisting of at least about 10 ng/ml of IL-6 and at least about 1 ng/ml of G-CSF.

2. The method of claim 1, wherein the mediator is at least about 10 ng/ml of IL-6.

3. The method of claim 1, wherein the mediator is at least about 1 ng/ml of G-CSF.

4. The method of claim 1, wherein the mediator is at least about 10 ng/ml of IL-6 and at least about 1 ng/ml of G-CSF.

5. The method of claim 1, wherein said bone-marrow stem cells are obtained from pigs of between about 2 days old and about 6 weeks old.

6. The method of claim 1, further comprising the step of maintaining said bone-marrow stem cells in the medium for at least about 30 days.
